(19) United States
(12) Patent Application Publication
(10) Pub. No.: US 2012/0003277 A1
(43) Pub. Date: Jan. 5, 2012

(54) NANOEMULSION VACCINES

(75) Inventors: James R. Baker, JR., Ann Arbor, MI (US); Tarek Hamouda, Milan, MI (US); Susan M. Ciotti, Ann Arbor, MI (US)

(73) Assignees: NANOBIO CORPORATION, Ann Arbor, MI (US); THE REGENTS OF THE UNIVERSITY OF MICHIGAN, Ann Arbor, MI (US)

(21) Appl. No.: 13/174,281

(22) Filed: Jun. 30, 2011

Related U.S. Application Data
(60) Provisional application No. 61/361,214, filed on Jul. 2, 2010.

(51) Int. Cl. A61K 9/107; A61K 39/145; A61K 39/245; A61K 39/155; A61K 39/21; A61K 39/29; A61K 39/125; A61K 39/235; A61K 39/23; A61K 39/285; A61K 39/215; A61K 39/07; A61K 39/10; A61K 39/106; A61K 39/118; A61K 39/08; A61K 39/112; A61K 39/09; A61K 39/085; A61K 39/095; A61K 39/102; A61K 39/108; A61K 39/104; A61P 37/04; A61P 31/04; A61P 31/14; A61P 31/16; A61P 31/18; A61P 31/20; A61P 31/22; A61K 39/00

(52) U.S. Cl. 424/400; 424/283.1; 424/209.1; 424/204.1; 424/234.1; 424/274.1; 424/231.1; 424/211.1; 424/208.1; 424/226.1; 424/227.1; 424/228.1; 424/230.1; 424/216.1; 424/233.1; 424/232.1; 424/218.1; 424/221.1; 424/246.1; 424/252.1; 424/261.1; 424/263.1; 424/247.1; 424/258.1; 424/244.1; 424/243.1; 424/249.1; 424/256.1; 424/257.1; 424/260.1

(57) ABSTRACT

The present invention provides methods and compositions for the stimulation of immune responses. Specifically, the present invention provides nanoemulsion compositions harboring one or more immunogens within the oil phase of the nanoemulsion and methods of using the same for the induction of immune responses (e.g., innate and/or adaptive immune responses (e.g., for generation of host immunity against an environmental pathogen)). Compositions and methods of the invention find use in, among other things, clinical (e.g., therapeutic and preventative medicine (e.g., vaccination)) and research applications.

This Application claims priority to U.S. Provisional Patent Application Ser. No. 61/361,214 filed 2 Jul. 2010, hereby incorporated by reference in its entirety.

### FIELD OF THE INVENTION

The present invention provides methods and compositions for the stimulation of immune responses. Specifically, the present invention provides nanoemulsion compositions harboring one or more immunogens within the oil phase of the nanoemulsion and methods of using the same for the induction of immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

### BACKGROUND

Immunization is a principal feature for improving the health of people. Despite the availability of a variety of successful vaccines against many common illnesses, infectious diseases remain a leading cause of health problems and death. Significant problems inherent in existing vaccines include the need for repeated immunizations, and the ineffectiveness of the current vaccine delivery systems for a broad spectrum of diseases.

In order to develop vaccines against pathogens that have been recalcitrant to vaccine development, and/or to overcome the failings of commercially available vaccines due to expense, complexity, and underutilization, new methods of antigen presentation must be developed which will allow for fewer immunizations, more efficient usage, and/or fewer side effects to the vaccine.

### SUMMARY OF THE INVENTION

The present invention provides methods and compositions for the stimulation of immune responses. In particular, the present invention provides nanoemulsion compositions harboring one or more immunogens within the oil phase of the nanoemulsion and methods of using the same for the induction of immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

In some embodiments, the nanoemulsion compositions harboring one or more immunogens within the oil phase of the nanoemulsion comprises oil, a cationic surfactant, water, an organic solvent and one or more immunogens present within the internal (oil) phase of the emulsion. Thus, the invention provides specific nanoemulsion compositions wherein an immunogen (e.g., antigenic substance described herein) mixed therewith resides within the internal (oil) phase of the emulsion. In a preferred embodiment, the invention provides a composition comprising an emulsion and an immunogen, the emulsion comprising an aqueous phase, an oil phase, and a solvent, wherein the immunogen is located within the internal (oil) phase of the emulsion, wherein the presence of immunogen in the internal phase of the emulsion provides a composition that is more immunogenic than an emulsion mixed with immunogen wherein the immunogen is not located within the internal oil phase of the emulsion (e.g., a composition comprising an emulsion wherein immunogen is located within the internal (oil) phase of the emulsion provides enhanced mucosal immunity after administration to a subject compared to mucosal immunity induced by an emulsion mixed with immunogen wherein the immunogen is not located within the internal oil phase of the emulsion after administration to a subject). In a preferred embodiment, the presence of ethanol within the emulsion stabilizes the emulsion/immunogen composition (e.g., thereby making the immunogen/emulsion composition more immunogenic than emulsion/immunogen composition lacking ethanol). Although an understanding of a mechanism is not needed to practice the invention, and the invention is not limited to any particular mechanism of action, in some embodiments, the solvation of the oil phase due to the presence of ethanol facilitates location of immunogen within the oil phase of the emulsion (e.g., thereby stabilizing the emulsion/immunogen composition (e.g., leading to enhanced uptake and/or delivery of the immunogen to antigen presenting cells (e.g., dendritic cells) when administered to a subject)). In another preferred embodiment, alcohol (e.g., ethanol) presence within an emulsion/immunogen composition of the invention participates in and/or is causative of the localization of immunogen within the oil phase of the emulsion (e.g., in the absence of alcohol (e.g., ethanol), immunogen does not localize within the oil phase of the emulsion). In yet another preferred embodiment, a nanoemulsion composition harboring one or more immunogens within the oil phase of the nanoemulsion does not contain any (e.g., any detectable level of) bacterial toxin, endotoxin and/or cytokine. In another preferred embodiment, a nanoemulsion composition harboring one or more immunogens within the oil phase of the nanoemulsion does not penetrate below the basement membrane of the nasal mucosa. In yet another preferred embodiment, a nanoemulsion composition harboring one or more immunogens within the oil phase of the nanoemulsion does not transit to the olfactory bulb.

A variety of nanoemulsion compositions are described herein that find use in the present invention. The present invention is not limited to a particular oil present in the nanoemulsion. A variety of oils are contemplated, including, but not limited to, soybean, avocado, squalene, olive, canola, corn, rapeseed, safflower, sunflower, fish, flavor, and water insoluble vitamins. The present invention is also not limited to a particular organic solvent. A variety of solvents are contemplated including, but not limited to, an alcohol (e.g., including, but not limited to, methanol, ethanol, propanol, and octanol), glycerol, polyethylene glycol, and an organic phosphate based solvent. In a preferred embodiment, the solvent is ethanol. Nanoemulsion components including oils, solvents and others are described in further detail below.

In some embodiments, the emulsion further comprises a surfactant. The present invention is not limited to a particular surfactant. A variety of surfactants are contemplated including, but not limited to, nonionic and ionic surfactants (e.g., TRITON X-100; TWEEN 20; TWEEN 80 and TYLOXAPOL).

In certain embodiments, the emulsion further comprises a cationic halogen containing compound. The present invention is not limited to a particular cationic halogen containing compound. A variety of cationic halogen containing compounds are contemplated including, but not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, and tetradecyltrimethylammonium halides. The present invention is also not limited to a particular halide. A variety of halides are contemplated including, but not limited to, halide selected from the group consisting of chloride, fluoride, bromide, and iodide.

In still further embodiments, the emulsion further comprises a quaternary ammonium containing compound. The present invention is not limited to a particular quaternary ammonium containing compound. A variety of quaternary ammonium containing compounds are contemplated including, but not limited to, Alkyl dimethyl benzyl ammonium chloride, dialkyl dimethyl ammonium chloride, n-Alkyl dimethyl benzyl ammonium chloride, n-Alkyl dimethyl ethylbenzyl ammonium chloride, Dialkyl dimethyl ammonium chloride, and n-Alkyl dimethyl benzyl ammonium chloride.

In still further embodiments, the emulsion further comprises a cationic surfactant. The present invention is not limited to a particular cationic surfactant. A variety of cationic surfactants are contemplated including, but not limited to dioloeyl-3-trimethylammonium propane (DOTAP) and dioleoyl-sn-glycerol-3-ethylphosphocholine (DEPC).

In some embodiments, the present invention provides a composition comprising a vaccine, the vaccine comprising an emulsion and an immunogen, the emulsion comprising an aqueous phase, an oil phase, and a solvent, wherein the immunogen is located within the internal (oil) phase of the emulsion. In some embodiment, the immunogen comprises a pathogen (e.g., an inactivated pathogen). In other embodiments, the immunogen comprises a pathogen product (e.g., including, but not limited to, a protein, peptide, polypeptide, nucleic acid, polysaccharide, or a membrane component derived from the pathogen). In some embodiments, the immunogen is inactivated prior to mixing with the emulsion. The present invention is not limited by the means by which the immunogen is inactivated. Means of inactivation include, but are not limited to, mixing with formaldehyde, heat inactivation, and mixing with an emulsion described herein. In some embodiments, a composition comprising an emulsion comprising an aqueous phase, an oil phase, and a solvent, protects and/or stabilizes an immunogen located within the internal (oil) phase of the emulsion (e.g., from degradation). In some embodiments, an alcohol present in the emulsion serves to solvate the oil (e.g., facilitating and/or allowing the immunogen to be localized within the oil phase of the emulsion). In a preferred embodiment, the presence of ethanol within the emulsion facilitates localization of immunogen within the oil phase of the emulsion (e.g., thereby stabilizing the emulsion/immunogen composition).

In some embodiments, the invention provides compositions and methods for the stimulation of immune responses. In particular, the present invention provides nanoemulsion adjuvant compositions and methods of using the same for the induction of immune responses (e.g., innate and/or adaptive immune responses (e.g., for generation of host immunity against an environmental pathogen)). In accordance with an aspect of the present invention, there is provided an immunogenic composition for eliciting an immune response in a host, including a human, the composition comprising a nanoemulsion adjuvant described herein and one or more immunogens, wherein the one or more immunogens are located within the oil phase of the emulsion.

In one aspect of the invention, there is provided a method of generating an immune response in a host, including a human, comprising administering thereto an immunogenic nanoemulsion adjuvant of the invention independently and/or in combination with one or more immunogens (e.g., antigenic (e.g., microbial pathogen (e.g., bacteria, viruses, etc.) protein, glycoprotein, lipoprotein, peptide, glycopeptide, lipopeptide, toxoid, carbohydrate, tumor-specific antigen) components). In some embodiments, a host immune response attained via administration of a nanoemulsion immunogen composition to a host subject is a humoral immune response. In some embodiments, a host immune response attained via administration of a nanoemulsion immunogen composition to a host subject is a cell-mediated immune response. In some embodiments, a host immune response attained via administration of a nanoemulsion immunogen composition to a host subject is an innate immune response. In some embodiments, a host immune response attained via administration of a nanoemulsion immunogen composition to a host subject is a combination of innate, cell-mediated and/or humoral immune responses and mucosal responses. In some embodiments, a composition comprising a nanoemulsion and one or more immunogens further comprises a pharmaceutically acceptable carrier.

In some embodiments of the invention, there is provided a kit for preparing an immunogenic nanoemulsion composition comprising an immunogen residing within the oil phase of the emulsion, comprising: (a) means for containing a nanoemulsion; and (b) means for containing at least one antigen/immunogen; and (c) means for combining the nanoemulsion and at least one antigen/immunogen to produce the immunogenic composition. The present invention provides several advantages over conventional adjuvants including, but not limited to, location of the immunogen within the internal (oil) phase of the emulsion (e.g., thereby facilitating delivery of the immunogen to antigen presenting cells (e.g., dendritic cells)), ease of formulation; effectiveness of adjuvanticity; lack of unwanted toxicity and/or host morbidity; and compatibility of antigens/immunogens with the adjuvant composition.

The present invention is not limited by the type of immunogen (e.g., antigenic component (e.g., pathogen, pathogen component, antigen, immunogen, etc.)) that is utilized with (e.g., combined with and residing in the internal oil phase of the emulsion) a nanoemulsion of the invention. In certain embodiments, the antigen/immunogen is selected from the group consisting of virus, bacteria, fungus and pathogen products derived from the virus, bacteria, or fungus. The present invention is not limited to a particular virus. A variety of viral immunogens are contemplated including, but not limited to, influenza A and/or B virus, avian influenza virus, H5N1 influenza virus, H1N1 influenza virus, West Nile virus, SARS virus, Marburg virus, Arenaviruses, Nipah virus, alphaviruses, filoviruses, herpes simplex virus I, herpes simplex virus II, paramyxoviruses including but not limited to respiratory synthetial virus, sendai virus, sindbis virus, vaccinia virus, parvovirus, human immunodeficiency virus, hepatitis B virus, hepatitis C virus, hepatitis A virus, cytomegalovirus, human papilloma virus, picornavirus, hantavirus, junin virus, and ebola virus. The present invention is not limited to a particular bacteria. A variety of bacterial immunogens are contemplated including, but not limited to, Bacillus cereus, Bacillus circulars and Bacillus megaterium, Bacillus anthracis, bacterial of the genus Brucella, Vibrio cholera, Coxiella burnetii, Francisella tularensis, Chlamydia psittaci, Ricinus communis, Rickettsia prowazekii, bacteria of the genus Salmonella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis. The present invention is also not limited to a particular fungus. A variety of fungal immunogens are contemplated including, but not limited to, Candida and Aspergillus.

In some embodiments, a nanoemulsion provided herein skews an immune response toward a Th1 type response. In some embodiments, a nanoemulsion provided herein skews an immune response toward a Th2 type response. In some embodiments, a nanoemulsion provided herein skews an immune response toward a Th17 type response. In some embodiments, a nanoemulsion provided herein provides a balanced Th1/Th2 response and/or polarization (e.g., an IgG subclass distribution and cytokine response indicative of a balanced Th1/Th2 response). Thus, a variety of immune responses may be generated and/or measured in a subject administered a nanoemulsion of the present invention including, but not limited to, activation, proliferation and/or differentiation of cells of the immune system (e.g., B cells, T cells, dendritic cells, antigen presenting cells (APCs), macrophages, natural killer (NK) cells, etc.); up-regulated or down-regulated expression of markers and/or cytokines; stimulation of IgA, IgM, and/or IgG titers; splenomegaly (e.g., increased spleen cellularity); hyperplasia, mixed cellular infiltrates in various organs, and/or other responses (e.g., of cells) of the immune system that can be assessed with respect to immune stimulation known in the art.

In some embodiments, administering comprises contacting a mucosal surface of the subject with the nanoemulsion immunogen composition. The present invention is not limited by the mucosal surface contacted. In some preferred embodiments, the mucosal surface comprises nasal mucosa. In some embodiments, the mucosal surface comprises vaginal mucosa. In some embodiments, administrating comprises parenteral administration. The present invention is not limited by the route chosen for administration of a nanoemulsion of the present invention. In some embodiments, inducing an immune response primes the immune system of a host to respond to (e.g., to produce a Th1 and/or Th2 type response (e.g., thereby providing protective immunity) one or more pathogens (e.g., B. anthracis, vaccinia virus, C. botulinum, Y pestis and/or HIV, etc.) in the host subject (e.g., human or animal subject). In some embodiments, the immunity comprises systemic immunity. In some embodiments, the immunity comprises mucosal immunity. In some embodiments, the immune response comprises increased expression of IFN-γ and/or TNF-α in the subject. In some embodiments, the immune response comprises a systemic IgG response. In some embodiments, the immune response comprises a mucosal IgA response.

In some embodiments, the present invention provides a method of determining the type of immune response that will be generated in a host post administration of nanoemulsion comprising providing a nanoemulsion and characterizing the nanoemulsion (e.g., characterizing nanoemulsion, particle size, zeta potential (charge), and/or other properties) and correlating the properties of the nanoemulsion with the type of immune response that will be generated in the host. In some embodiments, a nanoemulsion (e.g., alone or in combination with an antigen/immunogen) is identified as stable and in the presence of an antigen/immunogen is used to induce a desired immune response in a receipient host. In some embodiments, a nanoemulsion (e.g., alone or in combination with one or more antigens (e.g., whole cell pathogen or component thereof)) with a zeta potential above 30 mV is used to induce a desired immune response in a host administered the same. However, the present invention is not so limited. In some embodiments, a nanoemulsion (e.g., alone or in combination with one or more antigens (e.g., whole cell pathogen or component thereof)) with a zeta potential above about 2 mV, 5 mV, 10 mV, 15 mV, above 20 mV, above 25 mV, above 35 mV, or higher is identified as a nanoemulsion composition capable of inducing a desired immune response in a host administered the same. The present invention is not limited by the nature of the desired immune response. In some embodiments, the desired immune response is an acquired immune response in a host (e.g., a human host). In some embodiments, the desired immune response is a humoral immune response in a host (e.g., a human host). In some embodiments, the desired immune response is a cell-mediated immune response in a host (e.g., a human host). In some embodiments, the desired immune response is a combination of cell-mediated and/or humoral immune responses. In some embodiments, the desired immune response is a Th1 type immune response. In some embodiments, the desired immune response is a Th2 type immune response. In some embodiments, the desired immune response is a Th17 type immune response. In some embodiments, the present invention provides an immunogenic composition for eliciting an immune response in a host, including a human, the composition comprising: (a) at least one antigen and/or immunogen; and (b) a nanoemulsion, wherein the immunogen is located within the internal oil phase of the emulsion. In some embodiments, the composition comprises an adjuvant substance (e.g., a nanoemulsion adjuvant and/or a non-nanoemulsion adjuvant (e.g., CpG oligonucleotide or other adjuvant described herein)).

In yet another aspect of the invention, there is provided a method of modulating and/or inducing an immune response (e.g., toward and/or away from a Th1 and/or Th2 type response) in a subject (e.g., toward an antigen) comprising providing a host subject and a nanoemulsion composition of the invention, and administering the nanoemulsion to the host subject under conditions such that an immune response is induced and/or modulated in the host subject. In some embodiments, the host immune response is specific for the nanoemulsion. In some embodiments, the host immune response comprises enhanced expression and/or activity of Th1 type cytokines (e.g., IL-2, IL-12, IFN-γ and/or TNF-α, etc.) while concurrently lacking enhanced expression and/or activity of Th2 type cytokines (e.g., IL-4, IL-5, IL-10, etc.). In some embodiments, the host immune response comprises enhanced expression of Th2 type cytokines (e.g., IL-4, IL-5, IL-10, etc.) while concurrently lacking enhanced expression and/or activity of Th1 type cytokines (e.g., (e.g., IL-2, IL-12, IFN-γ and/or TNF-α, etc.). In some embodiments, a nanoemulsion adjuvant composition administered to a subject induces expression and/or activity of Th1-type cytokines that increases to a greater extent than the level of expression and/or activity of Th2-type cytokines. For example, in some embodiments, a subject administered a nanoemulsion composition induces a greater than 3 fold, greater than 5 fold, greater than 10 fold, greater than 20 fold, greater than 25 fold, greater than 30 fold or more enhanced expression of Th1 type cytokines (e.g., IL-2, IL-12, IFN-γ and/or TNF-α), with lower increases (e.g., less than 3 fold, less than two fold or less) enhanced expression of Th2 type cytokines (e.g., IL-4, IL-5, and/or IL-10). In some embodiments, a nanoemulsion composition administered to a subject induces expression and/or activity of Th2-type cytokines that increases to a greater extent than the level of expression and/or activity of Th1-type cytokines. For example, in some embodiments, a subject administered a nanoemulsion composition induces a greater than 3 fold, greater than 5 fold, greater than 10 fold, greater than 20 fold, greater than 25 fold, greater than 30 fold or more enhanced expression of Th2 type cytokines (e.g., IL-4, IL-5, and/or IL-10), with lower increases (e.g., less than 3 fold, less than two fold or less) enhanced expression of Th1 type cytokines (e.g., IL-2, IL-12, IFN-γ and/or TNF-α). In some embodiments, the host immune response comprises enhanced IL6 cytokine expression and/or activity while concurrently lacking enhanced expression and/or activity of other cytokines (e.g., IL4, TNF-α and/or IFN-γ) in the host. In some embodiments, the host immune response is specific for an antigen co-administered with the nanoemulsion. In some embodiments, administering the nanoemulsion to the host subject (e.g., in combination with an antigenic component (e.g., whole cell pathogen or component thereof)) induces and/or enhances the generation of one or more antibodies in the subject (e.g., IgG, IgM and/or IgA antibodies) that are not generated or generated at low levels in the host subject in the absence of administration of the nanoemulsion. In some embodiments, administering the nanoemulsion to the host induces a specific response to the nanoemulsion by epithelial cells of the host. In some embodiments, administering the nanoemulsion adjuvant to the host induces uric acid and/or inflamasome activation in the host (e.g., that is distinguishable from uric acid and/or inflamasome activation induced by other types of adjuvants (e.g., alum adjuvants).

Antigens and/or immunogens that may be included in an immunogenic nanoemulsion composition of the present invention, include, but are not limited to, microbial pathogens, bacteria, viruses, proteins, glycoproteins lipoproteins, peptides, glycopeptides, lipopeptides, toxoids, carbohydrates, and tumor-specific antigens. In some embodiments, mixtures of two or more antigens/immunogens may be utilized. Examples of immunogens and/or antigenic components of pathogens are described in detail herein.

In some embodiments, a nanoemulsion is formulated to comprise between 0.1 and 500 μg of a protein antigen (e.g., derived or isolated from a pathogen and/or a recombinant form of an immunogenic pathogen component). However, the present invention is not limited to this amount of protein antigen. For example, in some embodiments, more than 500 μg of protein antigen is present in a nanoemulsion for administration to a subject. In some embodiments, less than 0.1 μg of protein antigen is present in a nanoemulsion for administration to a subject. In some embodiments, about 5, about 10, about 15, about 20, about 25, about 30, about 35 or about 40 μg of protein antigen is present in a nanoemulsion (e.g., present in the internal oil phase of the emulsion) for administration to a subject. In some embodiments, a pathogen (e.g., a virus) is inactivated (e.g., by the nanoemulsion or by other means) and is then administered to the subject under conditions such that between about 10 and 10⁷ pfu (e.g., about 10², 10³, 10⁴, 10⁵, or 10⁶ pfu) of the inactivated pathogen is present in a dose administered to the subject. However, the present invention is not limited to this amount of pathogen present in a nanoemulsion administered. For example, in some embodiments, more than 10⁷ pfu of the inactivated pathogen (e.g., 10⁸ pfu, 10⁹ pfu, or more) is present in a dose administered to the subject.

In some embodiments, the present invention provides a composition comprising a 10% nanoemulsion solution. However, the present invention is not limited to this amount (e.g., percentage) of nanoemusion. For example, in some embodiments, a composition comprises less than 10% nanoemulsion (e.g., 9%, 8%, 7%, 6%, 5%, 4%, 3%, 2%, 1%, 0.5% or less). In some embodiments, a composition comprises more than 10% nanoemulsion (e.g., 15%, 20%, 25%, 30%, 35%, 40%. 45%, 50%, 55%, 60%, 65%, 70% or more). In some embodiments, a nanoemulsion of the present invention comprises any of the nanoemulsions described herein that are useful for generating a nanoemulsion composition comprising immunogen residing within the internal oil phase of the emulsion. In some embodiments, the nanoemulsion comprises W₂₀5EC. In some embodiments, the nanoemulsion comprises W₈₀5EC. In some embodiments, immune responses resulting from administration of a nanoemulsion adjuvant (e.g., individually and/or in combination with immunogenic pathogen components) protects the subject from displaying signs or symptoms of disease caused by a pathogen (e.g., influenza virus, vaccinia virus, B. anthracis, HIV, etc.).

In some embodiments, host immune responses resulting from administration of a nanoemulsion (e.g., individually and/or in combination with an antigenic/immunogenic component (e.g., whole cell pathogen or component thereof)) protects a subject from challenge with a subsequent exposure to live pathogen. In some embodiments, a nanoemulsion further comprises one or more adjuvants. The present invention is not limited by the type of adjuvant utilized. In some embodiments, the adjuvant is a CpG oligonucleotide. In some embodiments, the adjuvant is monophosphoryl lipid A. A number of other adjuvants that find use in the present invention are described herein. In some embodiments, the subject is a human. In some embodiments, immune responses resulting from administration of a nanoemulsion (e.g., individually and/or in combination with immunogenic pathogen components) reduces the risk of infection upon one or more exposures to a pathogen. In some embodiments, administration of a nanoemulsion to a host subject (e.g., in combination with an antigenic component (e.g., whole cell pathogen or component thereof)) induces the generation of one or more antibodies in the subject (e.g., IgG, IgM and/or IgA antibodies) that are not generated in the host subject in the absence of administration of the nanoemulsion adjuvant. In some embodiments, administration of a nanoemulsion composition harboring one or more immunogens within the oil phase of the nanoemulsion, wherein the dose of immunogen is a fraction of the dose utilized in a standard commercial antigen dose, provides the same or better stimulation of an immune response (e.g., generation antigen/immunogen-specific antibody titers) in a subject compared to the immune response elicited by the standard commercial antigen dose administered by itself (not in the context of a nanoemulsion composition harboring antigen within the oil phase of the nanoemulsion). In some embodiments, the antigen dose in a nanoemulsion composition harboring one or more immunogens within the oil phase of the nanoemulsion is the same as or less than (e.g., ½, ⅓, ¼, ⅕, ⅙ or less than) of the antigen dose needed to elicit a comparable immune response when the immunogen is not present in a nanoemulsion composition harboring antigen within the oil phase of the nanoemulsion.

The present invention also provides a composition for stimulating an immune response in a subject comprising a nanoemulsion and an immunogen wherein the immunogen resides within the internal oil phase of the emulsion, wherein the composition is configured to induce immunity to a pathogen from which the immunogen is derived in a subject. In some embodiments, the nanoemulsion comprises any nanoemulsion described herein. In some embodiments, the nanoemulsion comprises W₂₀5EC. In some embodiments, the nanoemulsion comprises W₈₀5EC. In some embodiments, the composition provides a subject between 1 and 500 μg of immunogen (e.g., recombinant immunogen (e.g., rPA, gp120) or inactivated immunogen) when administered to the subject. In some embodiments, a dose of the composition administered to a subject comprises between a 0.1% and 50% nanoemulsion solution (e.g., 5%, 10%, 20% or 40%). In some embodiments, the composition further comprises a pharmaceutically acceptable carrier. In some embodiments, a dose of the composition administered to a subject comprises a 1% nanoemulsion solution. In some embodiments, the immunogen is heat stable in the internal oil phase of the nanoemulsion adjuvant. In some embodiments, the composition is diluted prior to administration to a subject. In some embodiments, the subject is a human. In some embodiments, immunity is systemic immunity. In some embodiments, immunity is mucosal immunity.

### BRIEF DESCRIPTION OF DRAWINGS

FIG. 1 shows the components of 100% and 60% W₈₀5EC.

FIG. 2 shows a transmission electron micrograph of FLUZONE 2008-2009 vaccine. Three distinct structures are shown, corresponding to viral antigen particles contained in FLUZONE 2008-2009 vaccine (−25 nm (round), ˜100 nm (round), and ˜100 nm (crescent).

FIG. 3 shows transmission electron micrograph of a nanoemulsion, 5% W₈₀5EC mixed with 7.5 μg of FLUZONE 2008-2009 vaccine. The majority of viral antigen particles are associated with the nanoemulsion droplets.

FIG. 4 shows a transmission electron micrograph of a nanoemulsion, 20% W₈₀5EC mixed with 7.5 μg of FLUZONE. Viral antigen particles can be seen associated with the nanoemulsion droplets. (Circled areas are representative of viral antigen particles).

FIG. 5 shows Appearance Values for 5% W₈₀5EC+30 μg FLUZONE and 20% W₈₀5EC+30 μg FLUZONE at 0, 8, 24 and 48 hours Following Preparation.

FIG. 6 shows summary of Potency Values for Three Antigens Following SRID Testing of FLUZONE (30 μg), 5% W₈₀5EC+FLUZONE (30 μg) and 20% W₈₀5EC+FLUZONE (30 μg) at 0, 8, 24 and 48 hours Following Preparation.

FIG. 7 shows Particle Size Analysis by Dynamic Light Scattering (nm) and pH Results for 5% W₈₀5EC+FLUZONE(30 μg) and 20% W₈₀5EC+FLUZONE(30 μg) at 0, 8, 24 and 48 hours Following Preparation.

FIG. 8 shows W₈₀5EC-Adjuvant interaction with dendritic cells (DCs) in vitro.

FIG. 9 shows W₈₀5EC-Adjuvant mediates DC internalization of diverse antigen proteins.

FIG. 10 shows W₈₀5EC-adjuvant mediated antigen internalization by murine primary bone marrow derived dendritic cells.

FIG. 11 shows antigen uptake into nasal epithelium and lymphoid tissues.

FIG. 12 shows a comparison of particle size distribution profiles: 100% W₈₀5EC nanoemulsions (with ethanol; solid line) and 100% W₈₀5C (without ethanol; dotted line) after manufacture.

FIG. 13 shows particle size distribution profiles of 20% W₈₀5EC at initial (solid line) and one-week at 40° C./75% RH (dotted line).

FIG. 14 shows particle size distribution profiles of 20% W₈₀5C at time zero (solid line) and one-week at 40° C./75 RH (dotted line).

FIG. 15 shows the visual appearance of nanoemulsion formulations manufactured with (W₈₀5EC) and without (W₈₀5C) ethanol before and after ultracentrifugation at 30,000 G for 1 hour. Panel A: Prior to centrifugation. Panel B: After centrifugation. Panel C; After centrifugation.

FIG. 16 shows negative staining of 20% Nanoemulsions W₈₀5EC (left) and W₈₀5C viewed using transmission electron microscopy (TEM).

FIG. 17 shows negative staining of 20% W₈₀5C (4600 magnification).

FIG. 18 shows cross sectioned TEM images of 20% nanoemulsion formulated with ethanol, W₈₀5EC. 20% W₈₀5EC+HA antigens (light gray shapes) are found in the oil droplets (dark gray shapes). Left panel: 20% W₈₀5EC (Control); Right panel: 20% W₈₀5EC+30 μg total.

FIG. 19 shows cross sectioned TEM images of 20% nanoemulsion formulated without ethanol with 30 μg total HA where antigens (black particulates) are located outside the oil droplets (light gray circular shapes); left panel 13,500× magnification; right panel 34,000× magnification.

FIG. 20 shows particle size distribution of HBsAg, W₈₀5EC and HBsAg mixed with W₈₀5EC. Particle sizing: Size distribution was measured using a laser diffraction particle-sizer. Analysis of HBsAg alone (A), NE alone (B), and NE mixed with 10 g/ml of HBsAg (C). Data was processed and analyzed using Fraunhofer optical modeling and number weighted averaging (number %). Single population intensity peaks indicate monodisperse populations of HBsAg (28 nm), NE (349 nm), and HBsAg-NE (335 nm).

FIG. 21 shows design and Hemagglutination Inhibition (HAI) Geometric Mean Titers (GMT) and seroconversion rates in Ferret Study #1 following 1 and 2 doses of A/Wisconsin/67/2005 (H3N2) virus, with and without W₈₀5EC-nanoemulsion.

FIG. 22 shows viral titer in the nasal wash of ferrets vaccinated with different W₈₀5EC-adjuvanted vaccines in Ferret Study #1:

FIG. 23 shows viral titer in the nasal turbinates and lungs of ferrets vaccinated with different W₈₀5EC-adjuvanted vaccines in Ferret Study #1.

FIG. 24 shows the design of Ferret Study #2.

FIG. 25 shows A/Wisconsin (H₃N₂) HAI Titers and seroconversion in ferrets following a single intranasal dose of commercial vaccines ±20% W₈₀5EC-adjuvant in Ferret Study #2.

FIG. 26 shows HAI Titers and seroconversion to the three influenza vaccine strains in ferrets following a single intranasal dose of commercial vaccines ±20% W₈₀5EC-adjuvant in Ferret Study #2.

FIG. 27 shows HAI Titers and seroconversion to Wisconsin and other H₃N₂ influenza strains following two intranasal doses of commercial vaccines ±20% W₈₀5EC-Adjuvant (Day 48) in Ferret Study #2.

FIG. 28 shows HAI Titers and seroconversion to the three influenza vaccine strains in ferrets following a single intranasal dose of commercial vaccine FLUZONE (2007-2008) ±20% W₈₀5EC-Adjuvant in Ferret Study #3.

FIG. 29 shows HAI Titers and seroconversion to A/Wisconsin and other H3N2 influenza strains in ferrets following a single intranasal dose of commercial vaccine FLUZONE±20% W₈₀5EC-Adjuvant in Ferret Study #3.

FIG. 30 shows the design of FLUZONE (2008-2009) Commercial Vaccine +W₈₀5EC-Adjuvant Immunogenicity Study in Ferrets in Ferret Study #4:

FIG. 31 shows HAI GMT Against A/Brisbane 59 (H1N1) in Ferret Study #4.

FIG. 32 shows HAI GMT Against A/Brisbane 10 (H3N2) in Ferret Study #4.

FIG. 33 shows HAI GMT Against B/Florida in Ferret Study #4.

FIG. 34 shows HAI Titers and Seroconversion to the Three Influenza Vaccine Strains in Ferrets Following a Single Intranasal Dose of Commercial Vaccine FLUZONE (2008-2009) ±20% W₈₀5EC-Adjuvant (Day 28) in Ferret Study #4.

FIG. 35 shows HAI Titers and Seroconversion to the Three Influenza Vaccine Strains in Ferrets Following Two Intranasal Doses of Commercial Vaccine FLUZONE (2008-2009) ±20% W₈₀5EC-Adjuvant (Day 48) in Ferret Study #4.

FIG. 36 shows A/Wisconsin Hemagglutination Inhibition (HAI) Titers after Vaccination with W₈₀5EC-Adjuvanted Fluvirin® Vaccine (2007-2008) in New Zealand White Rabbits and Hartley Guinea Pigs.

FIG. 37 shows the average particle size (diameter) present in the ovalbumin/W₈₀5EC mixture post centrifugation.

FIG. 38 shows aqueous and oil phase ovalbumin concentrations of various ovalbumin/W₈₀5EC mixtures.

### DEFINITIONS

To facilitate an understanding of the present invention, a number of terms and phrases are defined below:

As used herein the term “microorganism” refers to microscopic organisms and taxonomically related macroscopic organisms within the categories of algae, bacteria, fungi (including lichens), protozoa, viruses, and subviral agents. The term microorganism encompasses both those organisms that are in and of themselves pathogenic to another organism (e.g., animals, including humans, and plants) and those organisms that produce agents that are pathogenic to another organism, while the organism itself is not directly pathogenic or infective to the other organism. As used herein the term “pathogen,” and grammatical equivalents, refers to an organism, including microorganisms, that causes disease in another organism (e.g., animals and plants) by directly infecting the other organism, or by producing agents that causes disease in another organism (e.g., bacteria that produce pathogenic toxins and the like).

As used herein the term “disease” refers to a deviation from the condition regarded as normal or average for members of a species or group, and which is detrimental to an affected individual under conditions that are not inimical to the majority of individuals of that species or group (e.g., diarrhea, nausea, fever, pain, and inflammation etc). A disease may be caused or result from contact by microorganisms and/or pathogens.

The terms “host” or “subject,” as used herein, refer to organisms to be treated by the compositions and methods of the present invention. Such organisms include organisms that are exposed to, or suspected of being exposed to, one or more pathogens. Such organisms also include organisms to be treated so as to prevent undesired exposure to pathogens. Organisms include, but are not limited to animals (e.g., humans, domesticated animal species, wild animals) and plants.

As used herein, the term “inactivating,” and grammatical equivalents, means having the ability to kill, eliminate or reduce the capacity of a pathogen to infect and/or cause a pathological responses in a host.

As used herein, the term “fusigenic” is intended to refer to an emulsion that is capable of fusing with the membrane of a microbial agent (e.g., a bacterium or bacterial spore). Specific examples of fusigenic emulsions include, but are not limited to, W₈₀8P described in U.S. Pat. Nos. 5,618,840; 5,547,677; and 5,549,901 and NP9 described in U.S. Pat. No. 5,700,679, each of which is herein incorporated by reference in their entireties. NP9 is a branched poly(oxy-1,2 ethaneolyl), alpha-(4-nonylphenal)-omega-hydroxy-surfactant. While not being limited to the following, NP9 and other surfactants that may be useful in the present invention are described in Table 1 of U.S. Pat. No. 5,662,957, herein incorporated by reference in its entirety.

As used herein, the term “lysogenic” refers to an emulsion that is capable of disrupting the membrane of a microbial agent (e.g., a bacterium or bacterial spore). In preferred embodiments of the present invention, the presence of both a lysogenic and a fusigenic agent in the same composition produces an enhanced inactivating effect than either agent alone. Methods and compositions (e.g., vaccines) using this improved antimicrobial composition are described in detail herein.

The term “emulsion,” as used herein, includes classic oil-in-water or water in oil dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase.

These other lipid structures include, but are not limited to, unilamellar, paucilamellar, and multilamellar lipid vesicles, micelles, and lamellar phases. Similarly, the term “nanoemulsion,” as used herein, refers to oil-in-water dispersions comprising small lipid structures. For example, in preferred embodiments, the nanoemulsions comprise an oil phase having droplets with a mean particle size of approximately 0.1 to 5 microns, although smaller and larger particle sizes are contemplated. The terms “emulsion” and “nanoemulsion” are often used herein, interchangeably, to refer to the nanoemulsions of the present invention.

As used herein, the terms “contacted” and “exposed,” refers to bringing one or more of the compositions of the present invention into contact with a pathogen or a subject to be protected against pathogens such that the compositions of the present invention may inactivate the microorganism or pathogenic agents, if present. The present invention contemplates that the disclosed compositions are contacted to the pathogens or microbial agents in sufficient volumes and/or concentrations to inactivate the pathogens or microbial agents.

The term “surfactant” refers to any molecule having both a polar head group, which energetically prefers solvation by water, and a hydrophobic tail that is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described by Meyers, (Meyers, Surfactant Science and Technology, VCH Publishers Inc., New York, pp. 231-245 (1992)), incorporated herein by reference. As used herein, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water that are good solubilizers of water in oils are at the low end of the scale.

As used herein, the term “germination enhancers” refer to compounds (e.g., amino acids (e.g., L-amino acids (L-alanine)), CaCl₂, Inosine, nitrogenous bases, etc.) that act, for example, to enhance the germination of certain strains of bacteria.

As used herein the term “interaction enhancers” refers to compounds that act to enhance the interaction of an emulsion with the cell wall of a bacteria (e.g., a Gram negative bacteria). Contemplated interaction enhancers include, but are not limited to, chelating agents (e.g., ethylenediaminetetraacetic acid (EDTA), ethylenebis(oxyethylenenitrilo)tetraacetic acid (EGTA), and the like) and certain biological agents (e.g., bovine serum abulmin (BSA) and the like).

The terms “buffer” or “buffering agents” refer to materials, that when added to a solution, cause the solution to resist changes in pH.

The terms “reducing agent” and “electron donor” refer to a material that donates electrons to a second material to reduce the oxidation state of one or more of the second material's atoms.

The term “monovalent salt” refers to any salt in which the metal (e.g., Na, K, or Li) has a net 1+ charge in solution (i.e., one more proton than electron).

The term “divalent salt” refers to any salt in which a metal (e.g., Mg, Ca, or Sr) has a net 2+ charge in solution.

The terms “chelator” or “chelating agent” refer to any materials having more than one atom with a lone pair of electrons that are available to bond to a metal ion.

The term “solution” refers to an aqueous or non-aqueous mixture.

As used herein, the term “therapeutic agent,” refers to compositions that decrease the infectivity, morbidity, or onset of mortality in a host contacted by a pathogenic microorganism or that prevent infectivity, morbidity, or onset of mortality in a host contacted by a pathogenic microorganism. Such agents may additionally comprise pharmaceutically acceptable compounds (e.g., adjutants, excipients, stabilizers, diluents, and the like). In some embodiments, the therapeutic agents (e.g., vaccines) of the present invention are administered in the form of topical emulsions, injectable compositions, ingestible solutions, and the like. When the route is topical, the form may be, for example, a spray (e.g., a nasal spray).

The terms “pharmaceutically acceptable” or “pharmacologically acceptable,” as used herein, refer to compositions that do not substantially produce adverse allergic or immunological reactions when administered to a host (e.g., an animal or a human). As used herein, “pharmaceutically acceptable carrier” includes any and all solvents, dispersion media, coatings, wetting agents (e.g., sodium lauryl sulfate), isotonic and absorption delaying agents, disintrigrants (e.g., potato starch or sodium starch glycolate), and the like.

As used herein, the term “topically” refers to application of the compositions of the present invention to the surface of the skin and mucosal cells and tissues (e.g., alveolar, buccal, lingual, masticatory, or nasal mucosa, and other tissues and cells which line hollow organs or body cavities).

As used herein, the term “topically active agents” refers to compositions of the present invention that illicit a pharmacological response at the site of application (contact) to a host.

As used herein, the term “systemically active drugs” is used broadly to indicate a substance or composition that will produce a pharmacological response at a site remote from the point of application or entry into a subject.

As used herein, the term “adjuvant” refers to an agent that increases the immune response to an antigen (e.g., a pathogen). A used herein, the term “immune response” refers to a subject's (e.g., a human or another animal) response by the immune system to immunogens (i.e., antigens) the subject's immune system recognizes as foreign. Immune responses include both cell-mediated immune responses (responses mediated by antigen-specific T cells and non-specific cells of the immune system) and humnasal immune responses (responses mediated by antibodies present in the plasma lymph, and tissue fluids). The term “immune response” encompasses both the initial responses to an immunogen (e.g., a pathogen) as well as memory responses that are a result of “acquired immunity.”

As used herein, the term “immunity” refers to protection from disease upon exposure to a pathogen Immunity can be innate (immune responses that exist in the absence of exposure to an antigen) and/or acquired (immune responses that are mediated by B and T cells following exposure to antigen and that exhibit specificity to the antigen).

As used herein, the term “immunogen” refers to an antigen that is capable of eliciting an immune response in a subject. In preferred embodiments, immunogens elicit immunity against the immunogen (e.g., a pathogen or a pathogen product) when administered in combination with a nanoemulsion of the present invention.

As used herein, the term “pathogen product” refers to any component or product derived from a pathogen including, but not limited to, polypeptides, peptides, proteins, nucleic acids, membrane fractions, and polysaccharides.

As used herein, the term “enhanced immunity” refers to an increase in the level of acquired immunity to a given pathogen following administration of a vaccine of the present invention relative to the level of acquired immunity when a vaccine of the present invention has not been administered.

As used herein, the term “purified” or “to purify” refers to the removal of contaminants or undesired compounds from a sample or composition. As used herein, the term “substantially purified” refers to the removal of from about 70 to 90%, up to 100%, of the contaminants or undesired compounds from a sample or composition.

As used herein, the term “surface” is used in its broadest sense. In one sense, the term refers to the outermost boundaries of an organism or inanimate object (e.g., vehicles, buildings, and food processing equipment, etc.) that are capable of being contacted by the compositions of the present invention (e.g., for animals: the skin, hair, and fur, etc., and for plants: the leaves, stems, flowering parts, and fruiting bodies, etc.). In another sense, the term also refers to the inner membranes and surfaces of animals and plants (e.g., for animals: the digestive tract, vascular tissues, and the like, and for plants: the vascular tissues, etc.) capable of being contacted by compositions by any of a number of transdermal delivery routes (e.g., injection, ingestion, transdermal delivery, inhalation, and the like).

As used herein, the term “sample” is used in its broadest sense. In one sense it can refer to animal cells or tissues. In another sense, it is meant to include a specimen or culture obtained from any source, such as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### DETAILED DESCRIPTION OF THE INVENTION

The present invention provides methods and compositions for the stimulation of immune responses. Specifically, the present invention provides nanoemulsion compositions harboring one or more immunogens within the oil phase of the nanoemulsion and methods of using the same for the induction of immune responses (e.g., innate and/or adaptive immune responses (e.g., for generation of host immunity against an environmental pathogen)). Compositions and methods of the present invention find use in, among other things, clinical (e.g. therapeutic and preventative medicine (e.g., vaccination)) and research applications.

The present invention is not limited to any mechanism of action. Indeed, an understanding of the mechanism is not necessary to practice the present invention. Nonetheless, it is contemplated that the nanoemulsion/immunogen compositions of the present invention, wherein the immunogen resides within the internal oil phase of the emulsion, elicits robust immune response against the immunogen due to, among other things, solvation of the oil phase by the organic solvent of the emulsion (e.g., that facilitates location of the immunogen to within the oil phase of the emulsion), stability of the immunogen within the oil phase of the emulsion, and/or enhanced uptake and delivery of the immunogen to antigen presenting cells (e.g., dendritic cells) facilitated by immunogen residing within the oil phase of the emulsion. In particular, emulsion immunogen compositions of the invention elicit robust mucosal immune responses (See e.g., Richter and Kipp, Curr Top Microbiol Immunol 240:159-76 (1999); Ruedl and Wolf, Int. Arch. Immunol., 108:334 (1995); and Mor et al., Trends Micrbiol 6:449-53 (1998) for reviews of the mucosal immune system). Mucosal antigens stimulate the Peyer's Patches (PP) of the gastrointestinal tract. The M cells of the PP then transport antigens to the underlying lymph tissue where they encounter B cells and initiate B cell development. IgA is secreted by primed B cells that have been induced to produce IgA by Th2 helper T cells. Primed B cells are transported throughout the lymph system where they populate all secretory tissues. IgAs are then secreted in mucosal tissues where they serve as a first-line defense against many viral and bacterial pathogens.

As shown in the Examples below, experiments conducted during the course of development of the present invention demonstrated that nanoemulsion immune compositions of the invention elicit both antibody responses as well as cytotoxic T cell responses. In a preferred embodiment, the invention provides a nanoemulsion-immunogen composition comprising an emulsion comprising an oil, cationic surfactant, water and an organic solvent, and an immunogen present within the internal oil phase of the emulsion (See, e.g., Examples 1-3). In a further preferred embodiment, the invention provides an emulsion comprising oil, cationic surfactant, water and ethanol mixed with a commercial vaccine (e.g., a commercial influenza vaccine (e.g., FLUZONE, sanofi pasteur, Swiftwater, Pa.)) approved for intramuscular administration in the United States) (See, e.g., Example 1). The present invention is not limited to this particular preparation. Indeed, any commercially available vaccine (e.g., influenza vaccine (e.g., seasonal influenza vaccine produced from year to year)) may be utilized in the compositions and methods of the invention. Each 0.5 mL of FLUZONE 2008-2009 commercial vaccine contains 45 g of total haemagglutinin (HA), in a ratio of 15 μg HA of each of the following 3 strains: A/Brisbane/59/2007 (H1N1), A/Uruguay/716/2007 (A/Brisbane/10/2007-like strain) (H3N2) and B/Florida/04/2006. Gelatin 0.05% is added as a stabilizer. Thimerosol is also contained in the multi-dose product presentation and each 0.5 mL contains 25 g mercury (thimerosol), <100 g formaldehyde, <0.02% Triton X-100 (t-octylphenoxypolyethoxyethanol) and <2% sucrose. As described in the Examples, an advantage of the immunogenic nanoemulsion immunogen compositions of the invention is the ability to use a significantly lower amount of antigen (e.g., 12 μg or 30 μg total HA/subject) in a composition while eliciting the same or better immune response than a full commercial dose of antigen in the absence of nanoemulsion (e.g., 45 g of HA). Although an understanding of the mechanism is not needed to practice the invention and the present invention is not limited to any particular mechanism of action, in some embodiments, the presence of the immunogen (e.g., HA) within the oil phase of the emulsion allows the composition comprising the emulsion and immunogen to elicit a more robust immune response than a composition comprising immunogen (e.g., HA) wherein the immunogen (e.g., HA) is not present in the oil phase of an emulsion. Thus, the invention also provides compositions and methods of using the same to elicit an immune response wherein the amounts of potentially undesirable components administered to a subject (e.g., thimerosol) are significantly reduced.

The nasally administered nanoemulsion vaccine compositions of the present invention have several advantages over parenterally administered vaccines. The vaccines can be easily administered when needed (e.g., immediately before or directly after exposure to the pathogen). When administered after exposure (e.g., after exposure of troops to a biological weapon), immune protection occurs specifically when needed. It is at this time that ongoing pathogen exposure might lead to infection. The administration methods of the present invention also avoid the need for expensive and problematic prophylactic vaccine programs. This approach provides the individual with specific immunity to the exact organisms exposed to, regardless of genetic or antigenic manipulation. The methods of the present invention are particularly valuable since they avoid the need for actual infection to induce immunity since even an attenuated infection can have undesired consequences. The present invention further provides methods of using nanoemulsions as adjuvants for parenteral administered vaccines. The present invention thus provides a rapid, killed vaccine for a range of naturally occurring and human administered pathological agents.

### Immunogenic Nanoemulsion Compositions

In some embodiments, the present invention provides immunogenic compositions (e.g., vaccines) comprising a nanoemulsion and one or more immunogens (e.g., inactivated pathogens and/or pathogen products), wherein the immunogen resides within the oil phase of the emulsion. The present invention provides immunogenic compositions (e.g., vaccines) for any number of pathogens. The present invention is not limited to any particular nanoemulsion formulation. Indeed, a variety of nanoemulsion formulations are contemplated (See e.g., below description and illustrative Examples and US Patent Application 20020045667, herein incorporated by reference).

### Immunogens

The immunogens (e.g., pathogens or pathogen products) and nanoemulsions of the present invention may be combined in any suitable amount utilizing a variety of delivery methods. Any suitable pharmaceutical formulation may be utilized, including, but not limited to, those disclosed herein. Suitable vaccine formulation may be tested for immunogenicity using any suitable method. For example, in some embodiments, immunogenicity is investigated by quantitating both antibody titer and specific T-cell responses. Nanoemulsion vaccines may also be tested in animal models of infectious disease states. Suitable animal models, pathogens, and assays for immunogenicity include, but are not limited to, those described below.

The present invention is not limited to the use of any one specific type of immunogen (e.g., inactivated pathogen, pathogen product, recombinant protein, etc.). Indeed, vaccines to a variety of pathogens are within the scope of the present invention. Accordingly, in some embodiments, the present invention provides vaccines to bacterial pathogens in vegetative or spore forms (e.g., including, but not limited to, Bacillus cereus, Bacillus circulans and Bacillus megaterium, Bacillus anthracia, Clostridium perfringens, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia enterocolitica, and Yersinia pseudotuberculosis). In other embodiments, the present invention provides vaccines to viral pathogens (e.g., including, but not limited to, influenza A & B, herpes simplex virus I, herpes simplex virus II, respiratory synthetial virus, sendai, sindbis, vaccinia, parvovirus, human immunodeficiency virus, hepatitis B, virus hepatitis C virus, hepatitis A virus, cytomegalovirus, and human papilloma virus, picornavirus, hantavirus, junin virus, and ebola virus). In still further embodiments, the present invention provides vaccines to fungal pathogens, including, but not limited to, Candida albicnas and parapsilosis, Aspergillus fumigatus and niger, Fusarium spp, Tiychophyton spp.

Bacteria for use in formulating the vaccines of the present invention can be obtained from commercial sources, including, but not limited to, American Type Culture Collection (ATCC). In some embodiments, bacteria are passed in animals prior to being mixed with nanoemulsions in order to enhance their pathogenicity for each specific animal host for 5-10 passages (Sinai et al., J. Infect. Dis., 141:193 (1980)). In some embodiments, the bacteria then are then isolated from the host animals, expanded in culture and stored at −80° C. Just before use, the bacteria are thawed and grown on an appropriate solid bacterial culture medium overnight. The next day, the bacteria are collected from the agar plate and suspended in a suitable liquid solution. The concentration of bacteria is adjusted so that the bacteria count is approximately 1.5×10⁸ colony forming units per ml (CFU/ml) based on the McFarland standard for bactericidal testing (Hendrichson and Krenz, 1991).

Viruses for use in formulating the vaccines of the present invention can be obtained from commercial sources, including, but not limited, ATCC. In some embodiments, viruses are passed in the prospective animal model for 5-10 times to enhance pathogenicity for each specific animal (Ginsberg and Johnson, Infect. Immun., 13:1221 (1976)). In some embodiments, the virus is collected and propagated in tissue culture and then purified using density gradient concentration and ultracentrifugation (Garlinghouse et al., Lab Anim Sci., 37:437 (1987); and Mahy, Br. Med. Bull., 41:50 (1985)). The Plaque Forming Units (PFU) are calculated in the appropriate tissue culture cells.

Lethal dose and/or infectious dose for each pathogen can be calculated using any suitable method, including, but not limited to, by administering different doses of the pathogens to the animals by the infective route and identifying the doses which result in the expected result of either animal sickness or death based on previous publications (Fortier et al., Infect Immun., 59:2922 (1991); Jacoby, Exp Gerontol., 29:89 (1994); and Salit et al., Can J Microbiol., 30:1022 (1984)).

### Pharmaceutical Compositions

The nanoemulsion vaccines of the invention may be formulated into pharmaceutical compositions that comprise the nanoemulsion vaccine in a therapeutically effective amount and suitable, pharmaceutically-acceptable excipients for pharmaceutically acceptable delivery. Such excipients are well known in the art.

By the phrase “therapeutically effective amount” it is meant any amount of the nanoemulsion vaccine that is effective in preventing, treating or ameliorating a disease caused by the pathogen associated with the immunogen administered in the composition comprising the nanoemulsion vaccine. By “protective immune response” it is meant that the immune response associated with prevention, treating, or amelioration of a disease. Complete prevention is not required, though is encompassed by the present invention. The immune response can be evaluated using the methods discussed herein or by any method known by a person of skill in the art.

Intranasal administration includes administration via the nose, either with or without concomitant inhalation during administration. Such administration is typically through contact by the composition comprising the nanoemulsion vaccine with the nasal mucosa, nasal turbinates or sinus cavity. Administration by inhalation comprises intranasal administration, or may include oral inhalation. Such administration may also include contact with the oral mucosa, bronchial mucosa, and other epithelia.

Exemplary dosage forms for pharmaceutical administration are described herein. Examples include but are not limited to liquids, ointments, creams, emulsions, lotions, gels, bioadhesive gels, sprays, aerosols, pastes, foams, sunscreens, capsules, microcapsules, suspensions, pessary, powder, semi-solid dosage form, etc.

The pharmaceutical compositions may be formulated for immediate release, sustained release, controlled release, delayed release, or any combinations thereof, into the epidermis or dermis. In some embodiments, the formulations may comprise a penetration-enhancing agent. Suitable penetration-enhancing agents include, but are not limited to, alcohols such as ethanol, triglycerides and aloe compositions. The amount of the penetration-enhancing agent may comprise from about 0.5% to about 40% by weight of the formulation.

The nanoemulsion vaccines of the invention can be applied and/or delivered utilizing electrophoretic delivery/electrophoresis. Further, the composition may be a transdermal delivery system such as a patch or administered by a pressurized or pneumatic device (i.e., “gene gun”).

Such methods, which comprise applying an electrical current, are well known in the art.

The pharmaceutical compositions for administration may be applied in a single administration or in multiple administrations.

If applied topically, the nanoemulsion may be occluded or semi-occluded. Occlusion or semi-occlusion may be performed by overlaying a bandage, polyoleofin film, article of clothing, impermeable barrier, or semi-impermeable barrier to the topical preparation.

An exemplary nanoemulsion adjuvant composition according to the invention is designated “W₈₀5EC” adjuvant. The composition of W₈₀5EC nanoemulsion is described in Example 1. This formulation contains ethanol as the organic solvent. The mean droplet size for the W₈₀5EC adjuvant is ˜400 nm. However, the present invention is not limited by the mean droplet size of the adjuvant. In some embodiments, the mean droplet size is <400 nm (e.g., in the range 120-400 nm). In some embodiments, the mean droplet size is >400 nm (e.g., in the range 400-800 nm). All of the components of the nanoemulsion are included on the FDA inactive ingredient list for Approved Drug Products.

The nanoemulsion adjuvants are formed by emulsification of an oil, purified water, nonionic detergent, organic solvent and surfactant, such as a cationic surfactant. An exemplary specific nanoemulsion adjuvant is designated as “60% W₈₀5EC”. The 60% W₈₀5EC-adjuvant is composed of the ingredients described in Example 1.

### Methods of Manufacture

The nanoemulsions of the invention can be formed using classic emulsion forming techniques. See e.g., U.S. 2004/0043041. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water or PBS, wherein one or more immunogens reside within the oil phase of the emulsion. The nanoemulsions of the invention are stable, and do not deteriorate even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures ranging from that of a semi-solid cream to that of a thin lotion, to that of a liquid and can be applied topically by any pharmaceutically acceptable method as stated above, e.g., by hand, or nasal drops/spray.

As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsion should harbor one or more immunogens in the oil phase of the emulsion if such one or more immunogens are mixed with the emulsion, and/or induce a protective immune response to a detectable level. Thus, in some embodiments, the invention provides an immunogenic composition comprising an emulsion and immunogen, wherein the immunogenic composition is characterized prior to its use, wherein characterizing the immunogenic composition comprises determining whether or not immunogen resides within the oil phase of the emulsion (e.g., using methods described in Examples 1-9), and selecting an immunogenic composition wherein immunogen resides within the oil phase of the emulsion. The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsion vaccine compositions of the present invention are not limited to any particular nanoemulsion. Any number of suitable nanoemulsion compositions may be utilized in the vaccine compositions of the present invention, including, but not limited to, those disclosed in Hamouda et al., J. Infect Dis., 180:1939 (1999); Hamouda and Baker, J. Appl. Microbiol., 89:397 (2000); and Donovan et al., Antivir. Chem. Chemother., 11:41 (2000), as well as those described herein. Preferred nanoemulsions of the present invention are those that comprise an oil, a surfactant (e.g., cationic surfactant), water, and an organic solvent (e.g., alcohol (e.g., ethanol)), and wherein the emulsion is formulated such that an immunogen mixed with the emulsion harbors the immunogen within the internal (oil) phase of the emulsion. Accordingly, preferred emulsion formulations utilize non-toxic solvents, such as ethanol. Although an understanding of the mechanism is not needed to practice the invention and the present invention is not limited to any particular mechanism of action, in some embodiments, the presence of an organic solvent (e.g., alcohol (e.g., ethanol)) participates in the solvation of the oil and localization of the immunogen within the oil phase of the emulsion.

In some preferred embodiments, the emulsions comprise (i) an aqueous phase; (ii) an oil phase; and (iii) one or more immunogens; wherein the one or more immunogens reside within the oil phase of the emulsion. In some embodiments, the emulsion comprises (iv) one or more additional compounds. In some embodiments, these additional compounds are admixed into either the aqueous or oil phases of the composition. In other embodiments, these additional compounds are admixed into a composition of previously emulsified oil and aqueous phases. In certain of these embodiments, one or more additional compounds are admixed into an existing emulsion composition immediately prior to its use. In other embodiments, one or more additional compounds are admixed into an existing emulsion composition prior to the compositions immediate use.

### Nanoemulsion Vaccines

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases.

The nanoemulsion and/or nanoemulsion vaccine of the present invention comprises droplets having an average diameter size, less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm.

### 1. Aqueous Phase

The aqueous phase can comprise any type of aqueous phase including, but not limited to, water (e.g., H₂O, distilled water, purified water, water for injection, de-ionized water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate buffered saline (PBS). The aqueous phase may further be sterile and pyrogen free.

### 2. Organic Solvents

Organic solvents in the nanoemulsion vaccines of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion vaccine include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

### 3. Oil Phase

The oil in the nanoemulsion vaccine of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, framesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

### 4. Surfactants

The surfactant in the nanoemulsion vaccine of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that contain a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂ CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis(imidazoyl carbonyl)), nonoxynol-9, Bis(polyethylene glycol bis(imidazoyl carbonyl)), BRIJ 35, BRIJ 56, BRIJ 72, BRIJ 76, BRIJ 92V, BRIJ 97, BRIJ 58P, CREMOPHOR EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN 20, SPAN 40, SPAN 60, SPAN 65, SPAN 80, SPAN 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, TRITON CF-21, TRITON CF-32, TRITON DF-12, TRITON DF-16, TRITON GR-5M, TRITON QS-15, TRITON QS-44, TRITON X-100, TRITON X-102, TRITON X-15, TRITON X-151, TRITON X-200, TRITON X-207, TRITON X-114, TRITON X-165, TRITON X-305, TRITON X-405, TRITON X-45, TRITON X-705-70, TWEEN 20, TWEEN 21, TWEEN 40, TWEEN 60, TWEEN 61, TWEEN 65, TWEEN 80, TWEEN 81, TWEEN 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products. Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quaternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H, 4H, 6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl)octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with an particular cationic containing compound. A variety of cationic surfactants are contemplated including, but not limited to dioloeyl-3-trimethylammonium propane (DOTAP) and dioleoyl-sn-glycerol-3-ethylphosphocholine (DEPC).

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecyl amine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, TRIZMA dodecyl sulfate, TWEEN 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion vaccine comprises a cationic surfactant, which can be cetylpyridinium chloride. In other embodiments of the invention, the nanoemulsion vaccine comprises a cationic surfactant, and the concentration of the cationic surfactant is less than about 5.0% and greater than about 0.001%. In yet another embodiment of the invention, the nanoemulsion vaccine comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion vaccine is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion vaccine is less than about 5.0% and greater than about 0.001%.

In another embodiment of the invention, the nanoemulsion vaccine comprises at least one cationic surfactant and at least one non-cationic surfactant. The non-cationic surfactant is a nonionic surfactant, such as a polysorbate (Tween), such as polysorbate 80, polysorbate 60 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the invention, the nanoemulsion vaccine comprises a cationic surfactant present in a concentration of about 0.01% to about 2%, in combination with a nonionic surfactant.

### 5. Additional Ingredients

Additional compounds suitable for use in the nanoemulsion vaccines of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion vaccine, or the additional compounds can be added to the original mixture to be emulsified. In certain of these embodiments, one or more additional compounds are admixed into an existing nanoemulsion composition immediately prior to its use.

Suitable preservatives in the nanoemulsion vaccines of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis(p-chlorophenyldiguanido)hexane), chlorphenesin (3-(−4-chloropheoxy)-propane-1,2-diol), Kathon C G (methyl and methylchloroisothiazolinone), parabens (methyl, ethyl, propyl, butyl hydrobenzoates), phenoxyethanol(2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion vaccine may further comprise at least one pH adjuster. Suitable pH adjusters in the nanoemulsion vaccine of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion vaccine can comprise a chelating agent. In one embodiment of the invention, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The nanoemulsion vaccine can comprise a buffering agent, such as a pharmaceutically acceptable buffering agent. Examples of buffering agents include, but are not limited to, 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, 5 M in H2O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H2O, Ammonium formate, ≧99.0% (calc. based on dry substance, NT), Ammonium oxalate monohydrate, ≧99.5% (RT), Ammonium phosphate dibasic solution, 2.5 M in H2O, Ammonium phosphate dibasic, ≧99.0% (T), Ammonium phosphate monobasic solution, 2.5 M in H2O, Ammonium phosphate monobasic, ≧99.5% (T), Ammonium sodium phosphate dibasic tetrahydrate, ≧99.5% (NT), Ammonium sulfate solution, for molecular biology, 3.2 M in H2O, Ammonium tartrate dibasic solution, 2 M in H2O (colorless solution at 20° C.), Ammonium tartrate dibasic, ≧99.5% (T), BES buffered saline, for molecular biology, 2× concentrate, BES, ≧99.5% (T), BES, for molecular biology, ≧99.5% (T), BICINE buffer Solution, for molecular biology, 1 M in H₂O, BICINE, ≧99.5% (T), BIS-TRIS, ≧99.0% (NT), Bicarbonate buffer solution, >0.1 M Na₂CO₃, >0.2 M NaHCO₃, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H₂O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H2O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2× concentrate, HEPES, ≧99.5% (T), HEPES, for molecular biology, ≧99.5% (T), Imidazole buffer Solution, 1 M in H2O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, ≧99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H2O, MOPS, ≧99.5% (T), MOPS, for luminescence, ≧99.5% (T), MOPS, for molecular biology, ≧99.5% (T), Magnesium acetate solution, for molecular biology, about 1 M in H2O, Magnesium acetate tetrahydrate, ≧99.0% (KT), Magnesium citrate tribasic nonahydrate, ≧98.0% (calc. based on dry substance, KT), Magnesium formate solution, 0.5 M in H2O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10× concentrate, piperazine, anhydrous, ≧99.0% (T), Potassium D-tartrate monobasic, ≧99.0% (T), Potassium acetate solution, for molecular biology, Potassium acetate solution, for molecular biology, 5 M in H2O, Potassium acetate solution, for molecular biology, about 1 M in H2O, Potassium acetate, ≧99.0% (NT), Potassium acetate, for luminescence, ≧99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H2O, Potassium formate solution, 14 M in H2O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, >99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, >95% (T), Potassium phthalate monobasic, ≧99.5% (T), Potassium sodium tartrate solution, 1.5 M in H2O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H2O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, .about.3 M in H2O, Sodium acetate trihydrate, ≧99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H2O, Sodium oxalate, ≧99.5% (RT), Sodium phosphate dibasic dihydrate, ≧99.0% (T), Sodium phosphate dibasic dihydrate, for luminescence, ≧99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H2O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H2O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H2O (colorless solution at 20.degree. C.), Sodium tetraborate decahydrate, ≧99.5% (T), TAPS, ≧99.5% (T), TES, ≧99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate—EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≧99.5% (NT), Triethanolamine, ≧99.5% (GC), Triethylamine, ≧99.5% (GC), Triethylammonium acetate buffer, volatile buffer, about.1.0 M in H2O, Triethylammonium phosphate solution, volatile buffer, .about.1.0 M in H2O, Trimethylammonium acetate solution, volatile buffer, .about.1.0 M in H2O, Trimethylammonium phosphate solution, volatile buffer, .about.1 M in H2O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, TRIZMA acetate, ≧99.0% (NT), TRIZMA base, ≧99.8% (T), TRIZMA base, ≧99.8% (T), TRIZMA base, for luminescence, ≧99.8% (T), TRIZMA base, for molecular biology, ≧99.8% (T), TRIZMA carbonate, >98.5% (T), TRIZMA hydrochloride buffer solution, for molecular biology, pH 7.2, TRIZMA hydrochloride buffer solution, for molecular biology, pH 7.4, TRIZMA hydrochloride buffer solution, for molecular biology, pH 7.6, TRIZMA hydrochloride buffer solution, for molecular biology, pH 8.0, TRIZMA hydrochloride, ≧99.0% (AT), TRIZMA hydrochloride, for luminescence, ≧99.0% (AT), TRIZMA hydrochloride, for molecular biology, ≧99.0% (AT), and TRIZMA maleate, ≧99.5% (NT). The nanoemulsion vaccine can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsion vaccines that may readily be diluted with water or another aqueous phase to a desired concentration without impairing their desired properties.

Some preferred embodiments of the invention employ an oil phase containing ethanol. For example, in some embodiments, the emulsions of the present invention contain (i) an aqueous phase and (ii) an oil phase containing ethanol as the organic solvent and optionally a germination enhancer, and (iii) polysorbate (TWEEN) as the surfactant (preferably about 5%). The invention is not limited by the type of polysorbate utilized. Indeed, a variety of polysorbate surfactants can be used including, but not limited to, TWEEN 20, TWEEN 60 and TWEEN 80.

In some other embodiments, the emulsions of the present invention comprise a first emulsion emulsified within a second emulsion, wherein (a) the first emulsion comprises (i) an aqueous phase; and (ii) an oil phase comprising an oil and an organic solvent; and (iii) a surfactant; and (b) the second emulsion comprises (i) an aqueous phase; and (ii) an oil phase comprising an oil and a cationic containing compound; and (iii) a surfactant.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles and any suitable pressurized spray device. Such spray bottles may be suitable for delivering the nanoemulsions intranasally or via inhalation.

These nanoemulsion-containing containers can further be packaged with instructions for use to form kits.

The present invention provides nanoemulsion/pathogen formulations suitable for use as vaccines. The compositions can be administered in any effective pharmaceutically acceptable form to subjects including human and animal subjects. Generally, this entails preparing compositions that are essentially free of pyrogens, as well as other impurities that could be harmful to humans or animals.

Particular examples of pharmaceutically acceptable forms include but are not limited to nasal, buccal, rectal, vaginal, topical or nasal spray or in any other form effective to deliver active vaccine compositions of the present invention to a given site. In preferred embodiments, the route of administration is designed to obtain direct contact of the compositions with the mucosal immune system (e.g., including, but not limited to, mucus membranes of the nasal and stomach areas). In other embodiments, administration may be by orthotopic, intradermal, subcutaneous, intramuscular or intraperitoneal injection. The compositions may also be administered to subjects parenterally or intraperitonealy. Such compositions would normally be administered as pharmaceutically acceptable compositions. Except insofar as any conventional pharmaceutically acceptable media or agent is incompatible with the vaccines of the present invention, the use of known pharmaceutically acceptable media and agents in these particular embodiments is contemplated. In additional embodiments, supplementary active ingredients also can be incorporated into the compositions.

For topical applications, the pharmaceutically acceptable carrier may take the form of a liquid, cream, foam, lotion, or gel, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly employed in pharmaceutical compositions for topical administration.

Actual amounts of compositions and any enhancing agents in the compositions may be varied so as to obtain amounts of emulsion and enhancing agents at the site of treatment that are effective in inactivating pathogens and producing immunity. Accordingly, the selected amounts will depend on the nature and site for treatment, the desired response, the desired duration of biocidal action and other factors. Generally, the emulsion compositions of the invention will comprise at least 0.001% to 100%, preferably 0.01 to 90%, of emulsion per ml of liquid composition. It is envisioned that the formulations may comprise about 0.001%, about 0.0025%, about 0.005%, about 0.0075%, about 0.01%, about 0.025%, about 0.05%, about 0.075%, about 0.1%, about 0.25%, about 0.5%, about 1.0%, about 2.5%, about 5%, about 7.5%, about 10%, about 12.5%, about 15%, about 20%, about 25%, about 30%, about 35%, about 40%, about 50%, about 55%, about 60%, about 65%, about 70%, about 75%, about 80%, about 85%, about 90%, about 95% or about 100% of emulsion per ml of liquid composition. It should be understood that a range between any two figures listed above is specifically contemplated to be encompassed within the metes and bounds of the present invention. Some variation in dosage will necessarily occur depending on the condition of the specific pathogen and the subject being immunized.

The person responsible for administration will, in any event, determine the appropriate dose for the individual subject. Moreover, for human administration, preparations should meet sterility, pyrogenicity, and general safety and purity standards as required by the FDA Office of Biologics standards.

The invention is further described by reference to the following examples, which are provided for illustration only. The invention is not limited to the examples, but rather includes all variations that are evident from the teachings provided herein. All publicly available documents referenced herein, including but not limited to U.S. patents are specifically incorporated by reference.

### EXAMPLES

The following examples serve to illustrate certain preferred embodiments and aspects of the present invention and are not to be construed as limiting the scope thereof.

In the experimental disclosure which follows, the following abbreviations apply: eq (equivalents); μ (micron); M (Molar); μM (micromolar); mM (millimolar); N (Normal); mol (moles); mmol (millimoles); μmol (micromoles); nmol (nanomoles); g (grams); mg (milligrams); μg (micrograms); ng (nanograms); L (liters); ml (milliliters); pi (microliters); cm (centimeters); mm (millimeters); μm (micrometers); nM (nanomolar); ° C. (degrees Centigrade); and PBS (phosphate buffered saline).

### Example 1

### Characterization of an Immunogenic Composition Comprising Nanoemulsion and Immunogen

Experiments were conducted during development of embodiments of the invention in order to characterize an immunogenic composition of the invention. An exemplary immunogenic composition was prepared, comprising nanoemulsion adjuvant (60% W₈₀5EC) mixed with FLUZONE 2008-2009 commercial vaccine (sanofi pasteur). This formulation was designated “NB-1008 Vaccine.”

The composition of 60% W₈₀5EC is Purified Water, Soybean Oil (super-refined), Dehydrated Alcohol (anhydrous ethanol), Polysorbate (Tween) 80 and Cetylpyridinium Chloride (CPC). All emulsion components meet USP/NF Pharmacopoeia compendial requirements and are included in the CDER Inactive Ingredients for Approved Drug Products database. In addition, all emulsion ingredients, except CPC are “generally recognized as safe” (GRAS) for oral administration at the proposed concentrations.

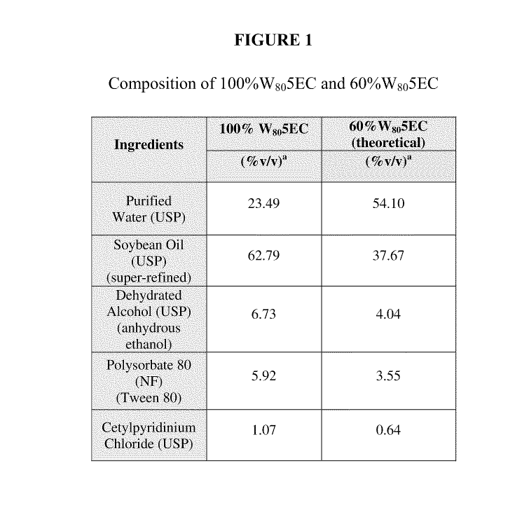

60% W₈₀5EC was prepared by a final dilution of a portion (7.5 kg) of 100% W₈₀5EC nanoemulsion with 5 kg of purified water. The quantitative composition of 100% W₈₀5EC and 60% W₈₀5EC is provided in FIG. 1 as % v/v.

NB-1008 is composed of a) commercial influenza vaccine (FLUZONE 2008-2009); the oil-in-water nanoemulsion 60% W₈₀5EC; and phosphate buffered saline (PBS) where indicated to achieve W₈₀5EC concentrations of 5, 10 and 20%.

60% W₈₀5EC is prepared by mixing the components using high shear homogenization followed by simple mixing. A specific ratio of excipients is necessary to create stable nanoemulsions. The use of high shear homogenization incorporates energy into the formulation to create nanometer-sized particles. The final concentration of 60% is achieved by dilution of 100% W₈₀5EC with purified water and simple mixing.

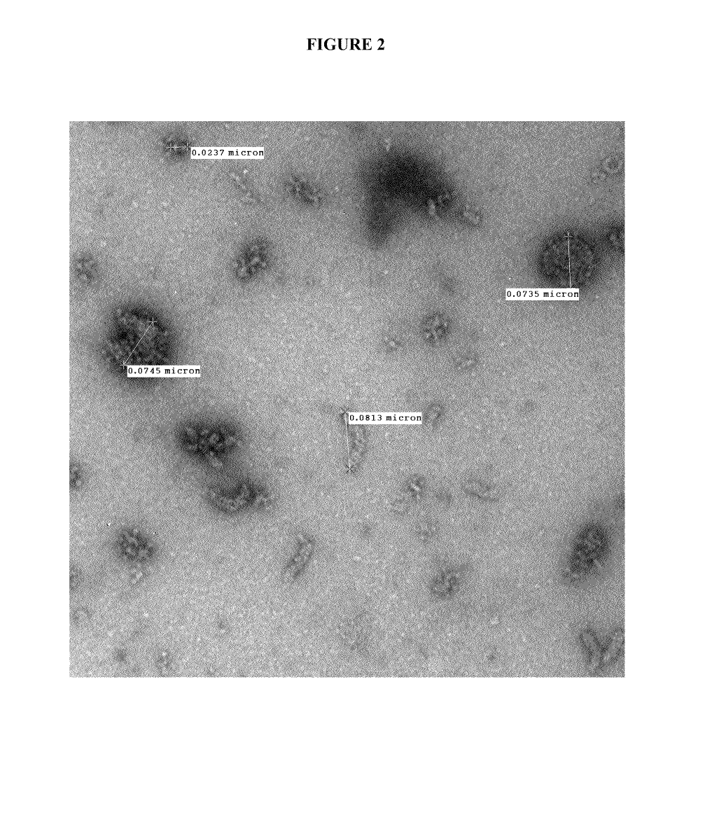

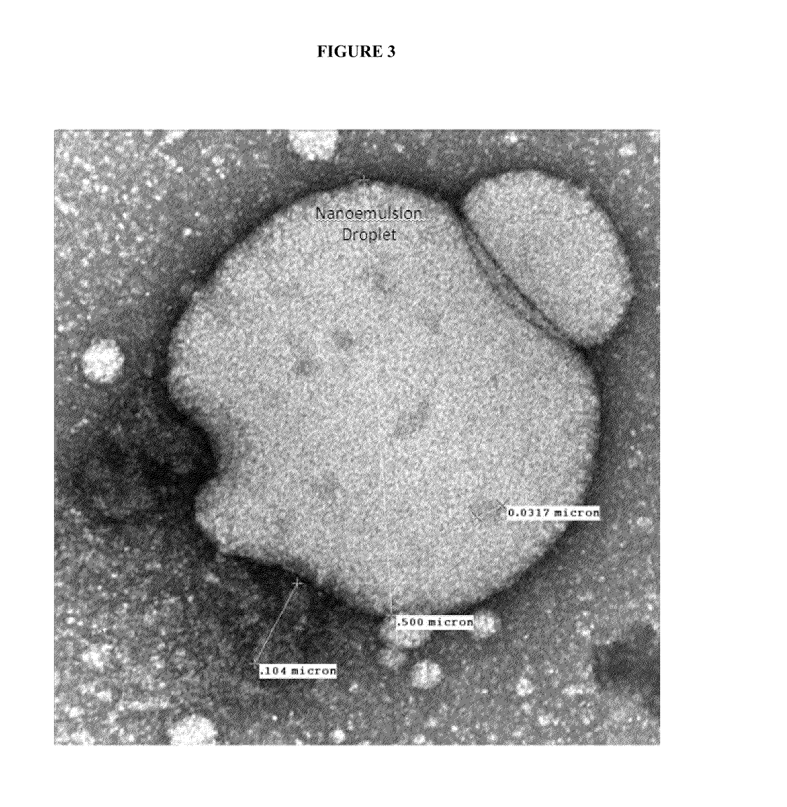

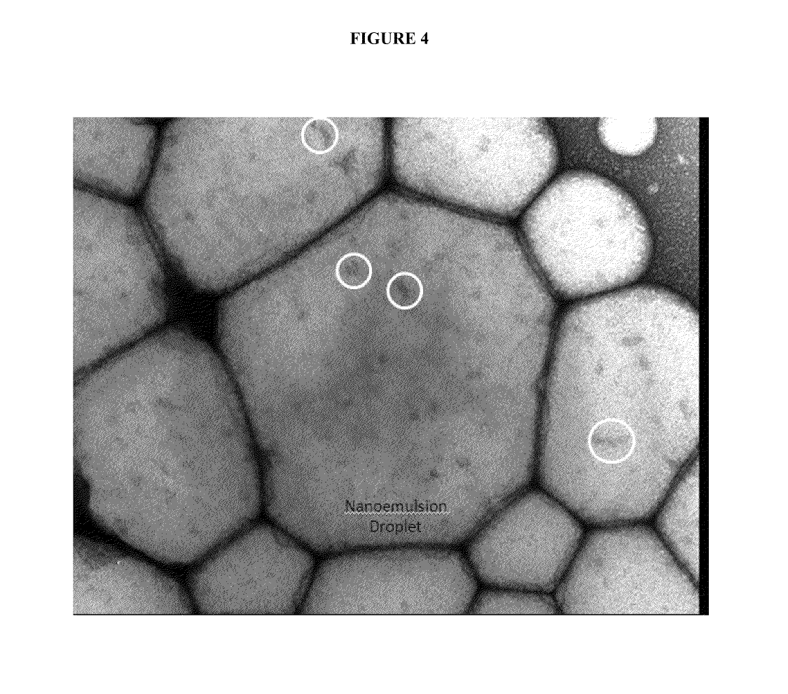

Three transmission electron micrographs are presented below in FIGS. 2-4. FIG. 2 depicts FLUZONE 2008-2009 vaccine. FIG. 3 shows 5% W₈₀5EC mixed with 7.5 μg of FLUZONE. FIG. 4 depicts 20% W₈₀5EC mixed with 7.5 μg of FLUZONE. Increasing the nanoemulsion concentration resulted in a greater physical association between the antigen and the nanoemulsion droplets.

Stability of FLUZONE (30 μg), 5% W₈₀5EC FLUZONE (30 μg) and 20% W₈₀5EC+FLUZONE (30 μg). The stability of FLUZONE (30 μg), 5% W₈₀5EC+FLUZONE (30 μg) and 20% W₈₀5EC+FLUZONE (30 μg) was assessed over 48 hours in order to provide support for extemporaneously prepared vaccines for their period of use (e.g., no more than 24 hours) and to determine if 5% or 20% W₈₀E5C interfered with the SRID assay of antigen potency.

Appearance, HA potency (of each of three antigens comprising FLUZONE A/Brisbane/59/2007 (H1N1), A/Uruguay/716/2007 (A/Brisbane/10/2007-like strain) (H₃N₂) and B/Florida/04/2006), pH and particle size were evaluated at 0, 8, 24 and 48 hours following preparation, with storage at 4° C. prior to dilution and plating.

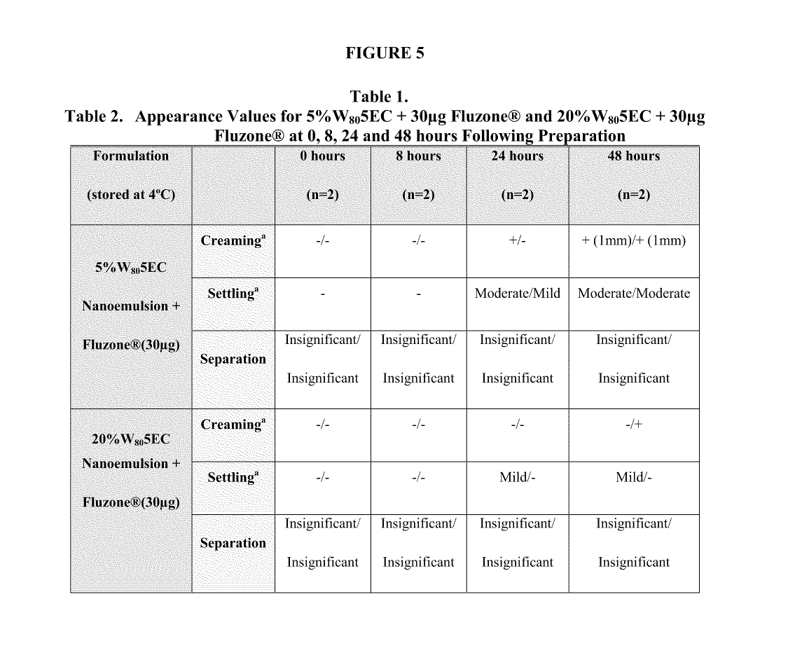

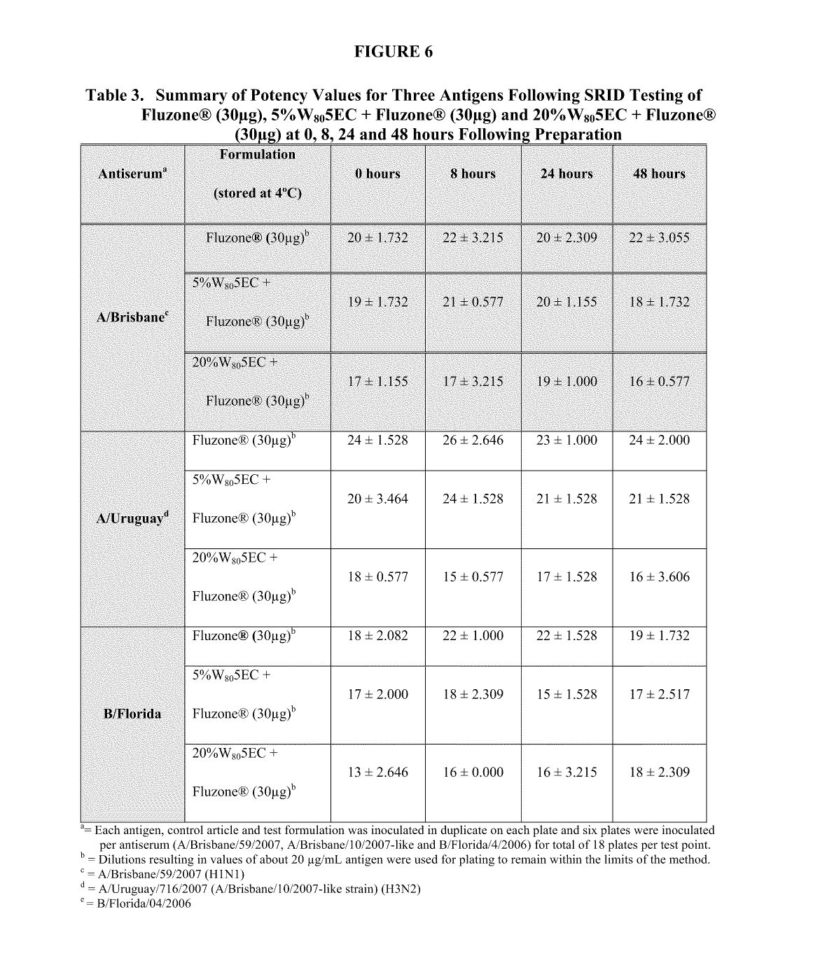

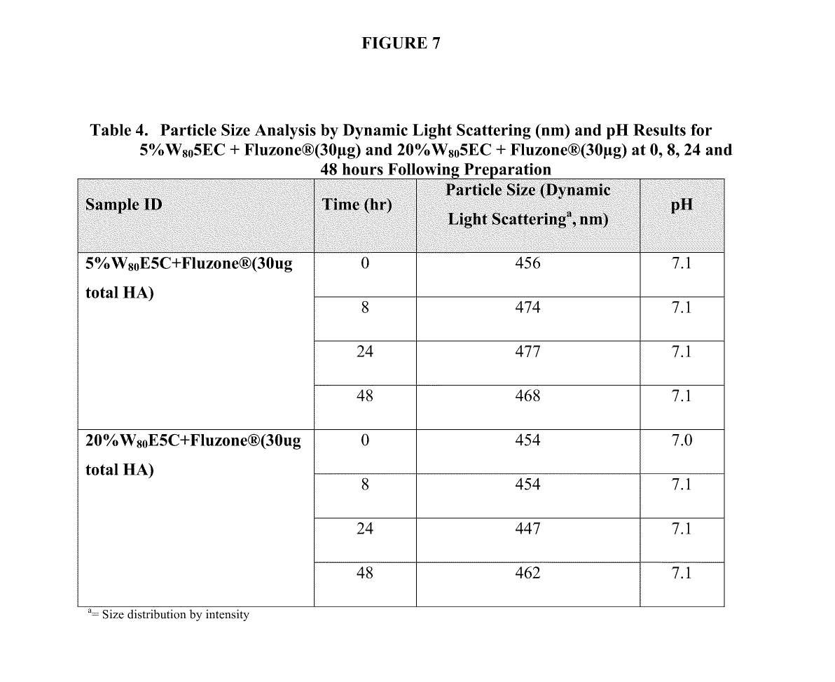

Results for appearance and potency of the two formulations, 5% W₈₀5EC+FLUZONE (30 μg) and 20% W₈₀5EC+FLUZONE (30 μg) are shown in FIGS. 5 and 6, respectively; results for Particle Size and pH are provided in FIG. 7.

Appearance values for 5% W₈₀5EC+30 μg FLUZONE and 20% W₈₀5EC+30 μg FLUZONE indicated stability over 48 hours, with slightly more creaming and settling observed at 24 and 48 hours post preparation, which is within acceptance criteria. The potency values for A/Brisbane/59/2007, A/Uruguay/716/2007, and B/Florida/4/2006 and were stable over 48 hours as shown in FIG. 6.

The presence of W₈₀5EC nanoemulsion at a concentration of 5% had no or little effect on the concentration of HA antigen for each virus strain as compared to the non-adjuvanted FLUZONE, on average, 20 μg/mL A/Brisbane, 21 μg/mL A/Uruguay and 17 μg/mL B/Florida and in the adjuvanted vaccine, compared to 21 μg/mL A/Brisbane, 24 μg/mL A/Uruguay and 20 μg/mL B/Florida in the non-adjuvanted vaccine).

In the formulation containing W₈₀5EC nanoemulsion at a concentration of 20% there was a slight decrease observed in potency, on average, 17 ug/mL A/Brisbane, 17 μg/mL A/Uruguay and 16 μg/mL B/Florida in the adjuvanted vaccine compared to 21 μg/mL A/Brisbane, 24 μg/mL A/Uruguay and 20 μg/mL B/Florida in the non-adjuvanted vaccine.

All particle size and pH results for 5% W₈₀5EC+FLUZONE (30 μg) and 20% W₈₀5EC+FLUZONE (30 μg) at 0, 8, 24 and 48 following preparation demonstrated stability at all time points (See, e.g., FIG. 7). Thus, the invention provides that the immunogenic compositions described herein are stable over 48 hours (e.g., as has been demonstrated in NB-1008 vaccines when stored at 4° C.).

### Example 2

### Adjuvant Properties of Nanoemulsion Comprising Immunogen Residing within Oil Phase

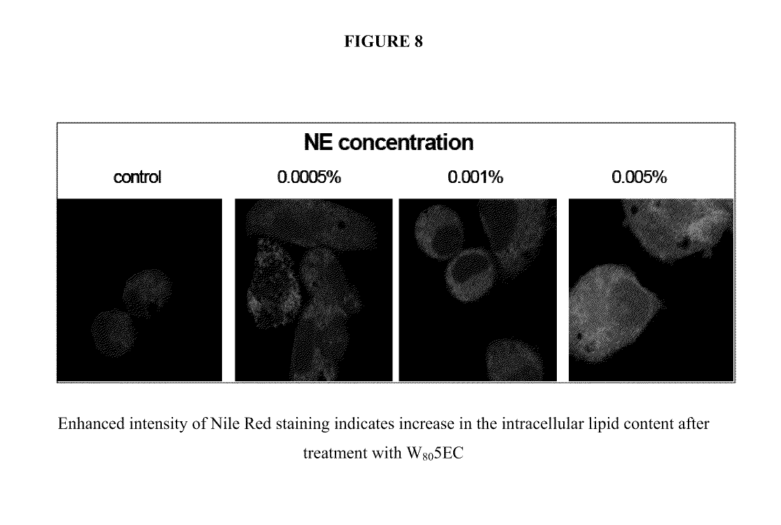

Nanoemulsion internalization into dendritic cells (DCs). DCs are specialized cells in the nasal epithelium designed to capture foreign substances and present them to the immune system in a manner that elicits specific protective immunity. Nanoemulsions increase the nasal mucosa residence time of the protein antigens residing within the oil phase of the emulsion. The size of the nanoemulsion droplets (≈400 nm) promotes uptake of the embedded antigen into DCs. Direct interactions of the nanoemulsion droplets with DCs are critical for this process to occur. Internalization of W₈₀5EC-adjuvant into dendritic cells was demonstrated using mouse JAWS II cells incubated with a range of W₈₀5EC-adjuvant concentrations. The changes in intracellular lipid content were detected by staining with the lipophilic dye, Nile Red, and analyzed using fluorescent imaging (See FIG. 8). The data demonstrated enhanced lipid content in cells incubated with W₈₀5EC, as compared to untreated controls. Internalization was also demonstrated using macrophages (RAW264.7).

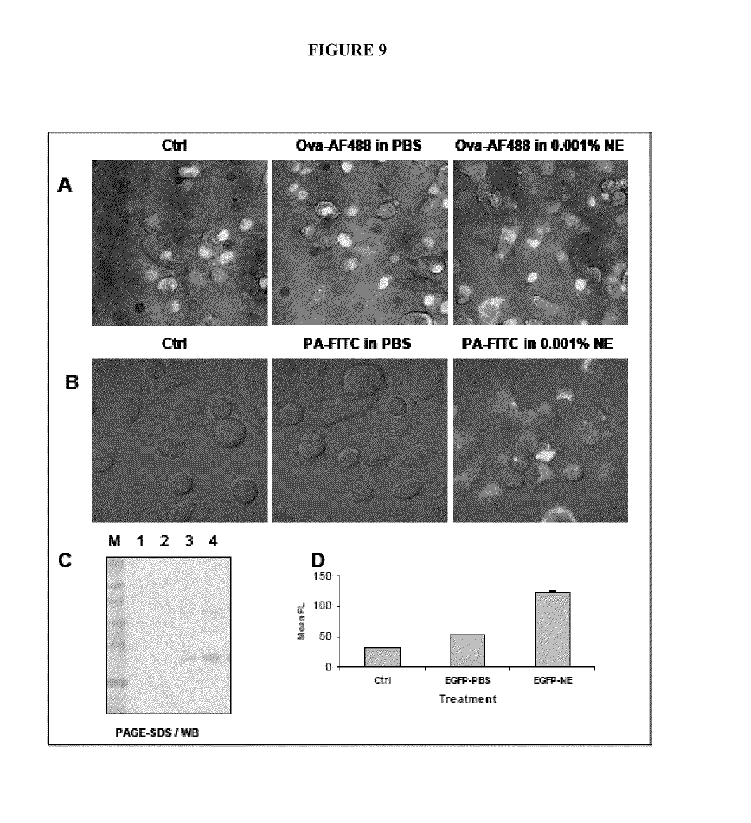

W₈₀5EC-adjuvant enhances antigen internalization into dendritic cells in vitro. Antigen uptake and subsequent presentation by dendritic cells plays a pivotal role in the initiation of immune response. The W₈₀5EC-adjuvant effect on antigen internalization was investigated in vitro in the murine DC line, JAWSII and in primary bone marrow-derived DCs (BMDC). As shown in FIG. 9, mixing with W₈₀5EC-adjuvant increased internalization of such diverse antigen proteins as ovalbumin (Ova), a recombinant protective antigen of anthrax (PA), recombinant hepatitis B virus surface antigen (HBsAg) and enhanced green fluorescent protein (EGFP) into dendritic cells JAWSII (See, e.g., FIGS. 9 A, B, C, and D, respectively). Panels A and B. JAWS II cells were incubated for 2 hours at 37° C. with either the fluorescently labeled ovalbumin-AlexaFluor488 (Ova-AF488, AlexaFluor 488) or PA (PA-FITC) mixed with 0.001% W₈₀5EC. The confocal microscopy images show significant intracellular green fluorescence in cultures treated with antigen+W₈₀5EC-adjuvant. The blue fluorescence indicates DAPI-stained cell nuclei (Panel A). Panel C. Western blot detection of hepatitis B surface antigen (HBsAg) uptake. Cell lysates were prepared from 1) untreated cells, 2) cells incubated with hepatitis B surface antigen alone, 3 and 4) cells incubated with HBsAg mixed with 0.001% and 0.005% W₈₀5EC, respectively. The main band of ˜22 kD represents HBsAg monomer detected with polyclonal anti-HBsAg IgG, followed by anti-IgG alkaline phosphatase-conjugated secondary antibody staining. Panel D. FACS analysis of EGFP internalization. Fluorescence values obtained for the untreated (Ctrl) and cells incubated with either EGFP alone (EGFP-PBS) or mixed with 0.001% W₈₀5EC (EGFP-NE).

The significant enhancement of antigen uptake was also observed in primary BMDCs.

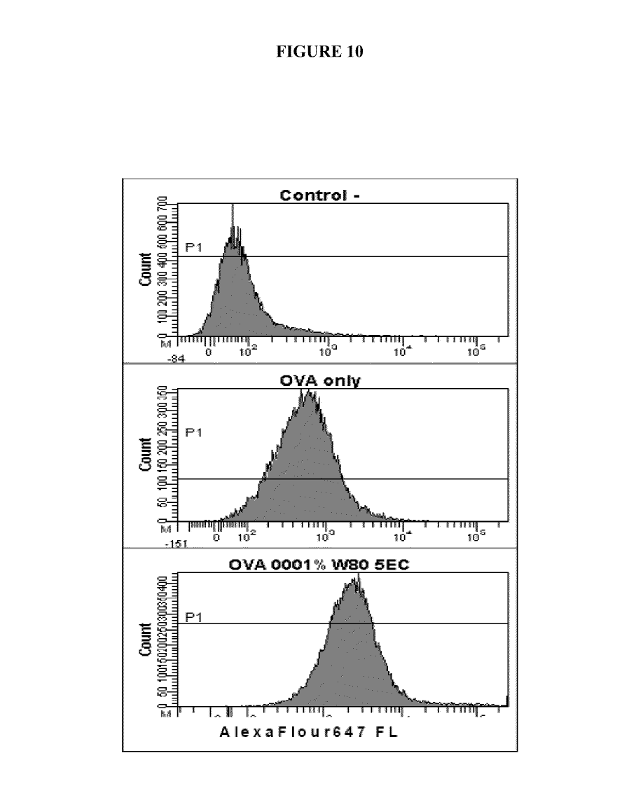

The Fluorescence Activated Cell Sorter (FACS) analysis of murine bone marrow-derived dendritic cells (BMDC) demonstrated that DCs treated with fluorescently labeled ovalbumin mixed with W₈₀5EC-adjuvant had 3- to5-fold (depending on W₈₀5EC concentration, data not shown) increased internalization of the antigen, as compared to the uptake of this protein alone (See FIG. 10). The BMDC were cultured in GM-CSF augmented medium for 6 days. The FACS analysis was performed with the untreated cells (Control) and with the BMDCs incubated for 2 hours at 37° C. with either the fluorescently labeled (AlexaFluor647) ovalbumin alone (OVA only) or with the ovalbumin mixed with 0.0001% W₈₀5EC-adjuvant (OVA-W₈₀5EC).

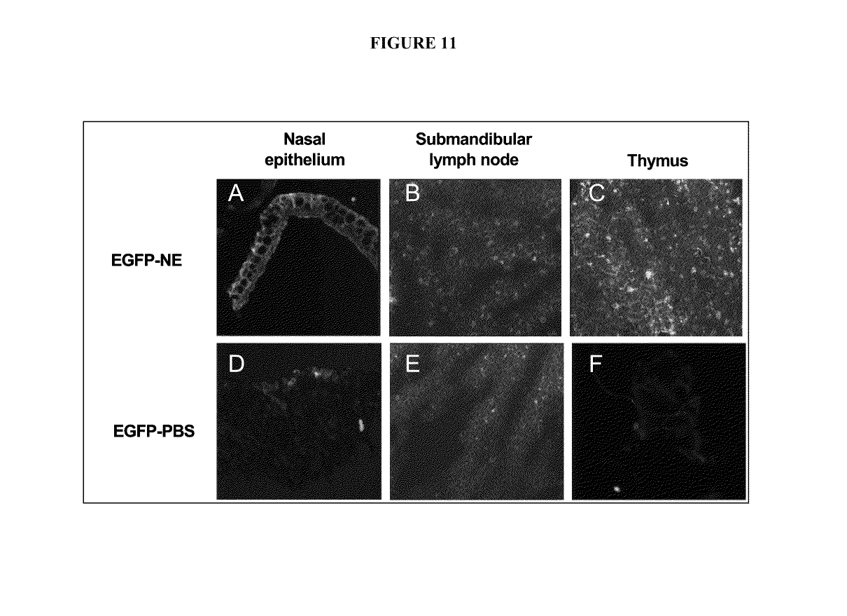

W₈₀5EC-adjuvant enhances antigen uptake in vivo. W₈₀5EC-adjuvant enhanced antigen uptake into the nasal mucosal epithelium and the lymphoid tissues. To examine the ability of W₈₀5EC to increase antigen uptake and trafficking in lymphoid tissues in vivo, the EGFP-W₈₀5EC-adjuvant (enhanced green fluorescent protein mixed with W₈₀5EC) mixtures were applied to the mouse nasal mucosa. At 24 hours post-treatment the green fluorescence was detected throughout the nasal epithelium, submandibular lymphoid tissue and thymus (See, e.g., FIGS. 11A, B, and C, respectively). The intense green fluorescence was detected in the majority of the epithelial cells (including the M cells) after administration of EGFP with W₈₀5EC-adjuvant, as compared to the less intense fluorescent signal seen when EGFP was delivered in PBS (See, e.g., FIGS. 11D, E and F, respectively). Antigen uptake into nasal epithelium and lymphoid tissues is enhanced when antigen is a component of W₈₀5EC-adjuvant. 10 μL of EGFP+W₈₀5EC (20 μg EGFP in 20% W₈₀5EC) was instilled into the nares of mice. Slides were prepared from tissues of animals euthanized 24 hours after treatment and EGFP fluorescence was analyzed using confocal microscopy. Images are presented at 200-fold magnification

### Example 3

### Physical Stability of Nanoemulsion Vaccine Adjuvants Containing Ethanol

The purpose of this example is to illustrate the stability of a nanoemulsion vaccine adjuvant containing ethanol at various time points. The composition of the 60% W₈₀5EC adjuvant is listed in Table 1.

Table 2 provides 3 month stability data for a nanoemulsion vaccine adjuvant according to the invention (60% W₈₀5EC nanoemulsion vaccine adjuvant).

TABLE 1
Composition of 60% W₈₀5EC-Adjuvant (w/w %)

| Ingredients | 60% W₈₀5EC |
|---|---|
| Purified Water, USP | 54.10% |
| Soybean Oil, USP | 37.67% |
| Dehydrated Alcohol, USP | 4.04% |
| (anhydrous ethanol) | |
| Polysorbate 80, NF | 3.55% |
| Cetylpyridinium Chloride, USP | 0.64% |

Tables 3 and 4 below, present stability data at 12 months and at 18 months, respectively, for a nanoemulsion vaccine according to the invention (60% W₈₀5EC nanoemulsion vaccine adjuvant). In addition, Table 5 provides data regarding antimicrobial effectiveness.

The nanoemulsion vaccine adjuvant was stable at all temperatures tested over the 12 month period and the nanoemulsion adjuvant was stable at refrigerated and room temperature for up to 18 months. There was moderate separation of the emulsion at 40 C at 18 months.

TABLE 2
3 month stability data for 60% W₈₀5EC-

| | Storage | | | Particle | Zeta | CPC Potency |
|---|---|---|---|---|---|---|
| Storage | Interval | | | Size, Mean | Potential | (Percent Label |
| Condition | (months)^{a} | Appearance | pH | (nm) | (mV)^{b} | Claim) |
| | Initial | Passes | 4.9 | 467 | 25.9 | 99.2, 100.5 |
| 5° C. | 1 | Passes | 5.2 | 468 | NT | 105.2, 106.0 |
| 5° C. | 3 | Passes | 5.2 | 422 | 41.7 | 105.1, 107.7 |
| 25° C. | 1 | Passes | 5.1 | 477 | NT | 107.3, 99.9 |
| 25° C. | 3 | Passes | 5.1 | 429 | 56.9 | 102.4, 105.4 |
| 40° C./75% RH | 1 | Passes | 5.0 | 450 | NT | 106.7, 108.3 |
| 40° C./75% RH | 3 | Passes | 4.1 | 419 | 48.9 | 97.8, 99.2 |

TABLE 3
Stability data for 60% W₈₀5EC at 12 months

| Storage | Appearance (Degree | | | | | | | |
|---|---|---|---|---|---|---|---|---|
| temp. | of separation) | | Particle size (nm) | | pH | | CPC (% recovery) | |
| (°) | Initial | 12 months | Initial | 12 months | Initial | 12 months | Initial | 12 months |
| 5 ± 3° C. | Pass | Pass | N/A | 437.6 | N/A | 5.22 | N/A | 108.1 |
| 22 ± 3° C. | Pass | Pass | 444 | 448.2 | 5.11 | 5.21 | 101.4 | 105.3 |
| 40 ± 2° C. | Pass | Pass | N/A | 447.7 | N/A | 4.71 | N/A | 99.8 |

TABLE 4
Stability data for 60% W₈₀5EC at 18 months

| Storage | Appearance (Degree | | | | | | | |
|---|---|---|---|---|---|---|---|---|
| temp. | of separation) | | Particle size (nm) | | pH | | CPC (% recovery) | |
| (°) | Initial | 18 months | Initial | 18 months | Initial | 18 months | Initial | 18 months |
| 5 ± 3° C. | Pass | Pass | N/A | 474.2 | N/A | 5.28 | N/A | 105.6 |
| 22 ± 3° C. | Pass | Pass | 444 | 469.45 | 5.11 | 5.11 | 101.4 | 99.9 |
| 40 ± 2° C. | Pass | moderate | N/A | 469.5 | N/A | 4.68 | N/A | 106.1 |

CPC recovery for 18 months is w/v

TABLE 5
Antimicrobial Effectiveness, Category 2 Products, USP<51>

| | S. aureus | P. aeruginosa | E. coli | C. Albicans | A. niger |
|---|---|---|---|---|---|
| Time = 0 | PASS | PASS | PASS | PASS | PASS |
| | More than 2.6 log | More than 4.5 log | More than 4.5 log | No increase from | No increase |
| | reduction from the | reduction from the | reduction from the | the initial | from the initial |
| | initial count at 14 | initial count at 14 | initial count at 14 | calculated count | calculated count |
| | days, and no | days, and no | days, and no | at 14 and 28 | at 14 and 28 |
| | increase from the | increase from the | increase from the | days. | days. |
| | 14 days' count at | 14 days' count at | 14 days' count at | | |
| | 28 days. | 28 days. | 28 days. | | |

### Example 4

### Comparison of Particle Size Stability of Nanoemulsion Vaccine Adjuvants with and Without Ethanol

The compostion of the nanoemulsion adjuvants formulated with (W₈₀5EC) and without ethanol (W₈₀5C) are presented in Table 6.

TABLE 6
Nanoemulsion (NE) Compositions formulated With Ethanol and Without Ethanol

| | 100% W₈₀5EC | 20% W₈₀5EC | 100% W₈₀5C | 20% W₈₀5C |
|---|---|---|---|---|
| | (b) | (c) | (d) | (e) |
| | With | With | Without | Without |
| | Ethanol | Ethanol | Ethanol | Ethanol |
| Excipients (a) | (w/w %) | (w/w %) | (w/w %) | (w/w %) |
| Purified Water, USP | 23.41 | 84.68 | 30.14 | 86.03 |
| Soybean Oil, USP-NP | 62.79 | 12.56 | 62.79 | 12.56 |
| Dehydrated Alcohol, USP | 6.73 | 1.346 | 0 | 0 |
| Tween 80, USP | 5.92 | 1.184 | 5.92 | 1.184 |
| Cetylpyridinium chloride | 1.068 | 0.214 | 1.068 | 0.214 |
| (CPC), USP | | | | |

The nanoemulsion adjuvants are formed by emulsification of an oil, purified water, nonionic detergent and surfactant, such as a cationic surfactant. An organic solvent such as ethanol may be added into the aqueous phase (water and cationic surfactant). The oil phase is then added to the aqueous phase to form the emulsion using a homogenizer. The emulsion is further processed to achieve the desired particle size

The formulations of 100% and 20% nanoemulsion with (W₈₀5EC) and without ethanol (W₈₀5C) were placed on stability at 5, 22 and 40° C./75RH at time 0 (initial) for 1 week, 2 weeks, 1 month, 3 months and 6 months. The Malvern Zetasizer was used to obtain the particle size distribution of the formulations

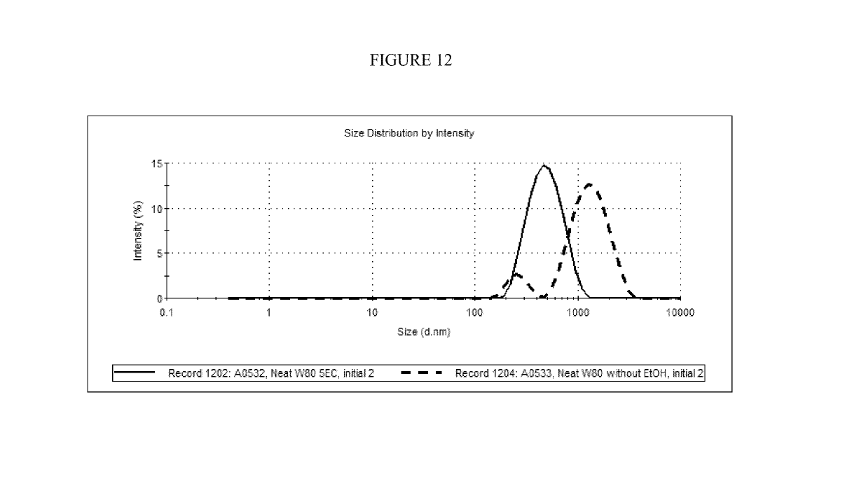

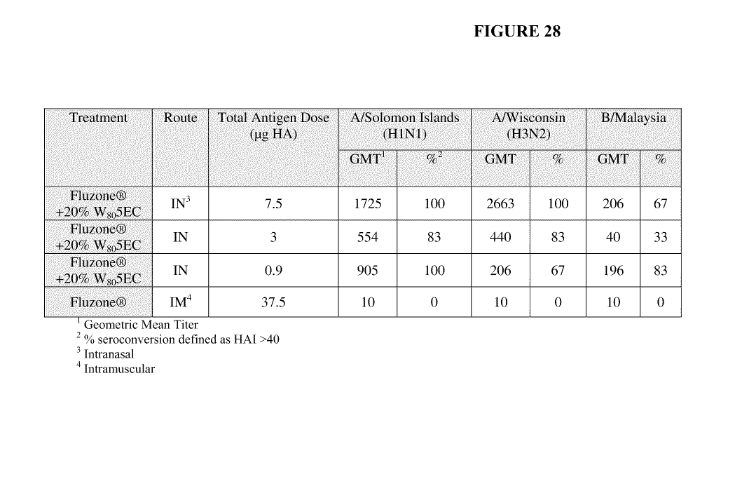

FIG. 28 compares the particle size distribution profile of the 100% W₈₀5EC (with ethanol) and W₈₀5C (without ethanol) at the initial time point (T=0). The W₈₀5C nanoemulsion shows a bi-modal distribution and has a larger mean particle size (927 nm) compared to the nanoemulsion containing ethanol. W₈₀5EC. The W₈₀5EC nanoemulsion, containing ethanol, has a uni-modal distribution and a mean particle size of 458 nm. A comparison of particle size distribution profiles is shown in FIG. 12.

Mean particle size, particle size range and polydispersity index of 20% W₈₀5EC and W₈₀5C nanoemulsion formulations manufactured at time zero and 1 week are summarized in Table 7. Mean particle size, particle size range and polydispersity index of 100% W₈₀5EC and W₈₀5C prepared at time zero and 1 week are summarized in Table 8.

TABLE 7
Mean particle size, range and polydispersity index of 20% W₈₀5EC
and W₈₀5C after one week storage at various storage conditions.

| | | | |
|---|---|---|---|
| | Mean Particle | Range | Polydispersity |
| Nanoemulsion | Size (nm) | (nm) | Index (PDI) |

20% (w/w) W₈₀5EC

| | | | |
|---|---|---|---|
| Initial | 457 ± 2.0 | 155-1284 | 0.12 ± 0.013 |
| 1 week at 5° C. | 467 ± 5.8 | 191-1358 | 0.13 ± 0.004 |
| 1 week at 22° C. | 460 ± 6.9 | 190-2775 | 0.15 ± 0.020 |
| 1 week at 40° C./75 RH | 463 ± 4.3 | 200-1349 | 0.15 ± 0.015 |

20% (w/w) W₈₀5C

| | | | |
|---|---|---|---|
| Initial | 880 ± 26.7 | 156-3946 | 0.29 ± 0.034 |
| 1 week at 5° C. | 934 ± 12.7 | 117-4174 | 0.29 ± 0.030 |
| 1 week at 22° C. | 951 ± 7.1 | 233-4900 | 0.29 ± 0.025 |
| 1 week at 40° C./75 RH | 944 ± 13.4 | 264-4900 | 0.33 ± 0.054 |

TABLE 8
Particle Size Profile of 100% W₈₀5EC and W₈₀5C: mean
particle size, range and polydispersity index after
one week storage at various storage conditions.

| | | | |
|---|---|---|---|
| | Mean Particle | Range | Polydispersity |
| Nanoemulsion | Size (nm) | (nm) | Index (PDI) |

100% (ww) W₈₀5EC

| | | | |
|---|---|---|---|
| Initial | 458 ± 5.7 | 205-1194 | 0.14 ± 0.047 |
| 1 week at 5° C. | 465 ± 5.9 | 182-1585 | 0.16 ± 0.013 |
| 1 week at 22° C. | 460 ± 6.9 | 190-2775 | 0.15 ± 0.020 |
| 1 week at 40° C./75 RH | 463 ± 4.3 | 200-1349 | 0.15 ± 0.015 |

100% (w/w) W₈₀EC

| | | | |
|---|---|---|---|
| Initial | 927 ± 17.3 | 153-2943 | 0.26 ± 0.043 |
| 1 week at 5° C. | 955 ± 28.6 | 174-5307 | 0.31 ± 0.019 |
| 1 week at 22° C. | 933 ± 16.7 | 250-4428 | 0.28 ± 0.021 |
| 1 week at 40° C./75 RH | 924 ± 23.1 | 216-4900 | 0.32 ± 0.030 |

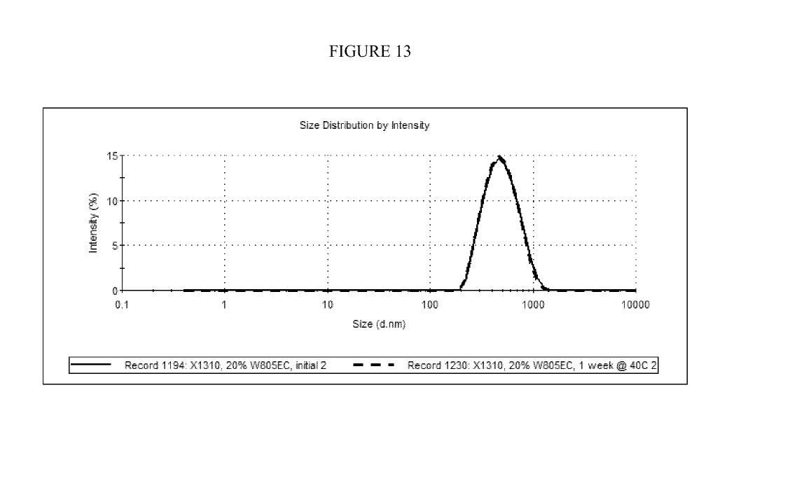

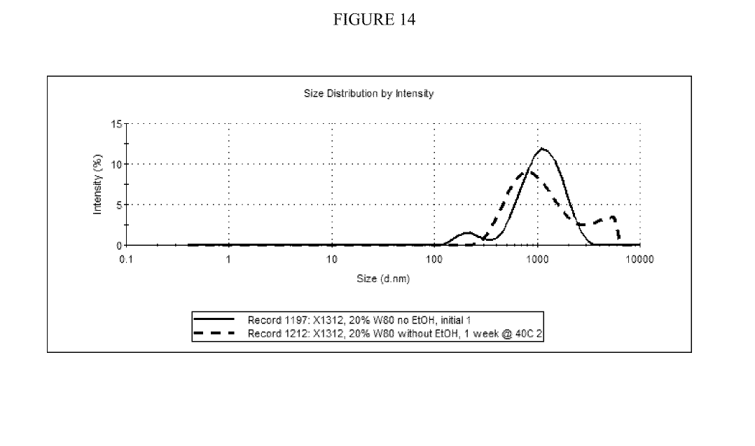

FIG. 13 shows a representative particle size distribution profile of the 20% W₈₀5EC at the initial time point (T=0) and 1 week at 40 C/75% RH. FIG. 14 shows a representative particle size distribution profile of the 20% W₈₀EC at time zero and 1 week at 40 C/75% RH. As observed in FIG. 14, nanoemulsions made without ethanol (W₈₀5C) appear unstable, as evidenced by increase in particle size over time. However, the particle size profiles of the W₈₀5EC formulation (FIG. 13) at time zero (initial) and 1 week at 40 C/75% RH are superimposable, indicating that this formulation is stable.

### Example 5

### Comparison of the Physical Appearance of Nanoemulsions Formulated with (W₈₀5Ec) and Without Ethanol (W₈₀Ec)

Dyes were added to representative nanoemulsion formulated with and without ethanol for purposes of visual comparison. Oil-red-0 (Solvent Red 27, Sudan Red 5B, C.I. 26125, C₂₆H₂₄N₄O), a fat-soluble dye, was added to help visualize the oil phase of the emulsion. Sodium fluorescein, a highly hydrophilic dye (D&C Yellow no. 8) was added to the formulation and due to its hydrophilic nature will solely partition into the hydrophilic (aqueous) phase of the nanoemulsion.

20% (w/w) W₈₀5EC and W₈₀EC containing 0.5% (w/w) Oil-Red-0 and 0.4% (w/v) sodium fluorescein were prepared to allow for visual comparison of the formulations. Oil-Red-0 powder was directly added to each 100% W₈₀5EC and W₈₀EC. The formulations were vortexed until the color of the formulation changed from white to pink. Each formulation was viewed under a light microscope (40×) to confirm the absence of solid red dye particles. Water was added to make a 40% nanoemulsion. Sodium fluorescein solution was then added to form the desired 20% nanoemulsion formulations.

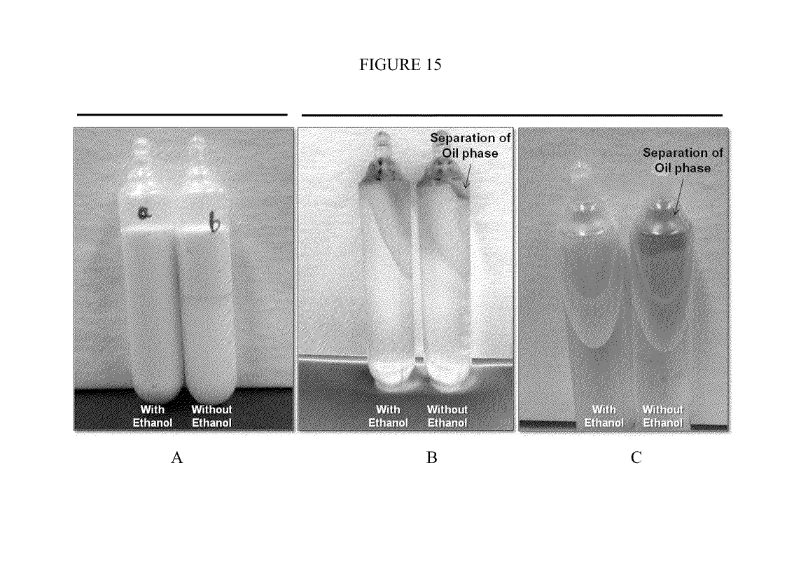

The centrifuge tube on the left side of Panel A in FIG. 15 contains 20% W₈₀5EC-dye formulation. This is a stable emulsion, as evidenced by the uniform pink color of the emulsion. The centrifuge tube of the right side of Panel A in FIG. 15 contains 20% W₈₀5C-dye formulation. Flocculation of the emulsion droplets is visible, as they have migrated to the top of the tube and the aqueous phase (pale yellow) is present below the flocculated emulsion.

20% nanoemulsion-dye formulations were also assessed for physical stability via ultracentrifugation. Centrifugal force is used to separate substances from each other based on their density. Thus, ultracentrifugation will separate the nanoemulsion droplets from the external aqueous phase of the formulation or from any unincorporated oil. Ultracentrifugation does not disrupt the structure of the nanoemulsion droplet (i.e. intact droplets remain in close proximity to each other) and does not cause coalescence of the droplets (fusion of nanoemulsion droplets). Due to the density of the droplets in the formulations, the emulsion droplets will move to the top of the tube and the aqueous phase will be below it. The 20% nanoemulsion-dye formulations (W₈₀5EC and W₈₀5C) were centrifuged at 30,000 G for 1 hour using a Beckman Ultracentrifuge

Following centrifugation, nanoemulsion droplets distribute to the top of the tube (white or pink layer) and the yellow-colored aqueous phase distributes below the droplet phase (FIG. 15, Panel B and C). Panel C from FIG. 15, illustrates that the oil has separated from the emulsion droplets (white-pink phase) in the formulation that does not contain ethanol. The top of the tube contains a dark oil phase (tube on right) that is not present in the formulation containing ethanol. This indicates instability of the nanoemulsion droplets that are formed in the absence of ethanol. In the formulation containing ethanol there is a continous emulsion droplet phase that resides above the external aqueous phase.

### Example 6

### Comparison of the Physical Appearance of Nanoemulsions Formulated with and without Ethanol Using Transmission Electron Microscopy

The physical appearance of nanoemulsions formulated with and without ethanol was assessed using transmission electron microscopy (TEM).

Negative staining was performed on the two prototype 20% nanoemulsion formulations, W₈₀5EC and W₈₀5C (Table 6). One to 5 microliters of each 20% nanoemulsion formulation was placed on a 300 mesh carbon-coated copper grid and stained with 1% (w/v) uranyl acetate in distilled and deionizer water (pH 7). Samples were viewed with a Philips CM-100 TEM equipped with a computer controlled compustage, a high resolution (2K×2K) digital camera and digitally imaged and captured using X-Stream imaging software (SEMTech Solutions, Inc., North Billerica, Mass.).

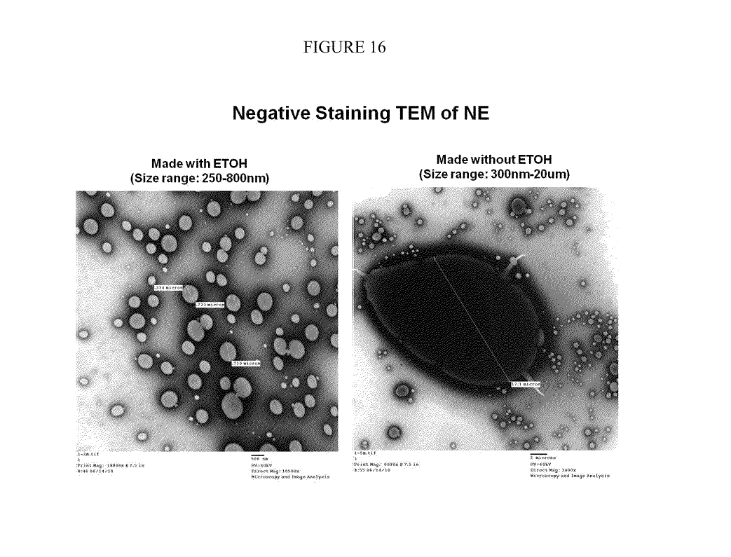

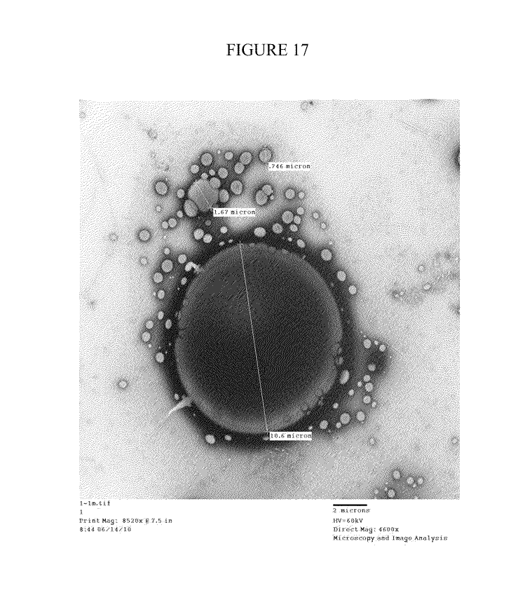

FIG. 16 illustrates that nanoemulsions formulated without ethanol form large emulsion droplets that are approximately 20 μm in diameter. This is in contrast to the small size of the droplets formed when nanoemulsions are prepared with ethanol, where there are no droplets greater than 1 μm. There is also evidence of instability in the formulation without ethanol, as coalescence of the droplets into larger oil droplets is evident (See FIG. 17).

### Example 7

### Localization of Influenza Antigen in Nanoemulsions Formulated with and without Ethanol

The localization of influenza antigen, 30 μg total of hemagglutinin antigen (HA; 10 μg HA/antigen) in the FLUZONE (2008-2009) vaccine, in 20% nanoemulsion formulations formulated with and without ethanol (Table 10) was determined using transmission electron microscopy (TEM). Each 0.5 mL of FLUZONE 2008-2009 commercial vaccine contains 45 g of total HA, in the recommended ratio of 15 μg HA of each of the following 3 strains: A/Brisbane/59/2007 (H1N1), A/Uruguay/716/2007 (A/Brisbane/10/2007-like strain) (H3N2) and B/Florida/04/2006.

TABLE 10
Composition of vaccines formulations containing 30 μg total of HA

| | 20% W₈₀5EC + 30 | 20% W₈₀5C + 30 | 20% W₈₀5EC |
|---|---|---|---|
| Components | mcg HA | mcg HA | (Control) |
| Purified Water | 18.03% | 19.38% | 18.03% |
| Soybean Oil | 12.56% | 12.56% | 12.56% |
| Dehydrated | 1.35% | 0% | 1.35% |
| Alcohol | | | |
| Polysorbate 80 | 1.18% | 1.18% | 1.18% |
| Cetylpyridinium | 0.213% | 0.213% | 0.213% |
| Chloride (USP) | | | |
| FLUZONE (2008-2009) | 66.67% | 66.67% | 0% |
| Phosphate | 0% | 0% | 66.67% |
| Buffered Saline (1X) | | | |
| Total (%) | 100.00 | 100.00 | 100.00 |

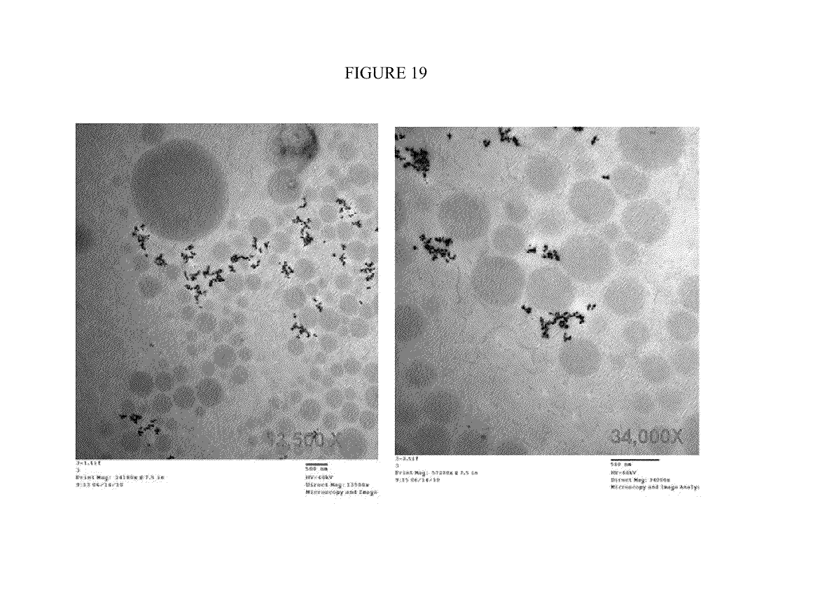

Description of TEM technique and sectioning technique. 20 μL of each nanoemulsion containing HA anatigen was fixed with 1% (w/v) osmium tetroxide. The fixed nanoemulsions were mixed with histogel in 1:10 ratio to form a solid mass. The solid mixture of nanoemulsion and histogel was sliced into thin 1 mm slices and rinsed with double distilled deionizer water. The cross-sectioned samples were dehydrated with ascending concentrations (30%-50%-70%-90%-100%-100%) of DURCUPAN (solution A) in double distilled deionizer water. These samples were transferred into embedding solution. The embedded samples were sectioned to a 75 nm thickness and placed on 300 mesh carbon-coated copper grid. The sections on the grids were stained with saturated uranyl acetate in distilled and deionizer water (pH 7) for 10 minutes followed by lead citrate for 5 minutes. The samples were viewed with a Philips CM-100 TEM equipped with a computer controlled compustage, a high resolution (2K×2K) digital camera and digitally imaged and captured using X-Stream imaging software (SEM Tech Solutions, Inc., North Billerica, Mass.). Cross sectioned TEM images of 20% W₈₀5C without ethanol with 30 μg total HA, show that the antigens (black particulates) are located outside the oil droplets (light gray circular shapes) (See FIG. 19).

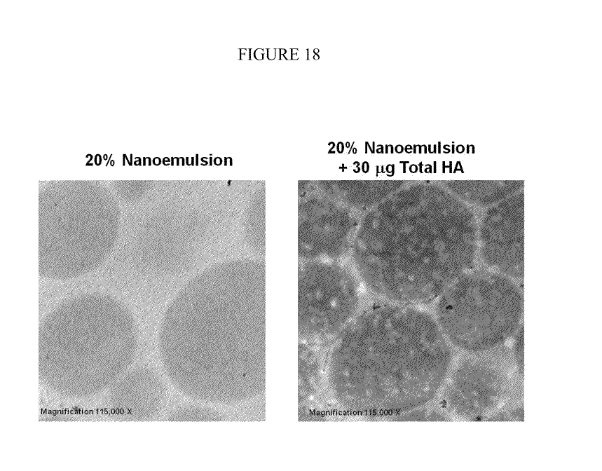

FIG. 18 shows cross section TEM images of the 20% W₈₀5EC with and without 30 μg total HA. The panel on the right illustrates that the HA antigens are located in the oil droplets. This is in contrast to the immunogen location in the absence of ethanol, where the HA antigens are not associated with the droplets (See FIG. 19). That is, the antigens are located outside of the droplets when the droplets are formed in the absence of ethanol.

### Example 8

### Localization of Antigen in W₈₀5EC Nanoemulsions as Assessed by Mean Particle Size of Vaccine Product

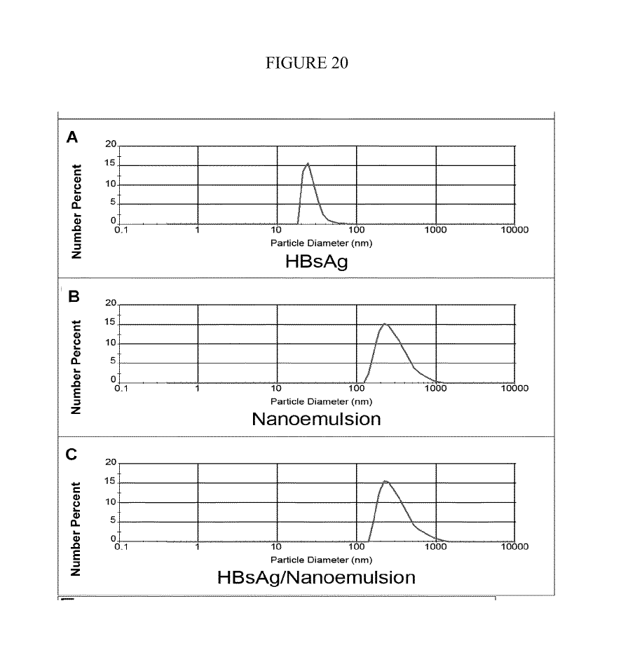

Experiments to assess antigen localization in the nanoemulsion were performed using dynamic light scattering. Hepatitis B surface antigen (HBsAG) was used as the antigen and W₈₀5EC was the nanoemulsion used in the experiment. Two peaks of different size were identified for 10 μg/mL hepatitis B surface antigen (HBsAg) and 20% W₈₀5EC before mixing (See FIGS. 20 and 20). However, after combining both components, only a single peak with a dynamic diameter of ≈300 nm was detected in the mixture (See FIG. 20C). The absence of two separate peaks indicated an association between the nanodroplet and HBsAg protein, and suggested that the antigen is in the oil droplet and not adsorbed/associated at the surface of the droplet, since the particle size distribution of the combination did not change (e.g., the mean particle size did not increase and there are also no particles less than 100 nm).

### Example 9

### Distribution of Ovalbumin in Emulsion W₈₀5EC

Experiments were conducted during development of embodiments of the invention in order to further characterize antigen partition within emulsion. As shown in Table 11 below, W₈₀5EC (20%) was tested with different amounts of ovalbumin (0.1 and 1.0 mg/ml) with phosphate buffered saline (DPBS), pH 7.4, at a molarity of either 5 millimolar (mM) or 25 mM.

TABLE 11
Ovalbumin-emulsion mixtures.

| | | | | | Volume (μl) | |
|---|---|---|---|---|---|---|
| | | Ovalbumin | 60% | | of the | |
| Sample | | at 2 mg/mL | W₈₀5EC | Sterilized | following | Final buffer |
| Number | Sample Group | (μl) | (μl) | Water (μl) | buffer | molarity (mM) |
| 1 | 0.1 mg/mL | 375 | 2500 | 3375 | 1x DPBS, | 25 |
| | Ovalbumin + | | | | 1250 | |
| | W805EC | | | | | |
| 2 | W₈₀5EC only | NA | | 3375 + 375 | | |
| 3 | 0.1 mg/mL | 375 | 2500 | 4375 | 1x DPBS, | 5 |
| | Ovalbumin + | | | | 250 | |
| | W₈₀5EC | | | | | |
| 4 | W₈₀5EC only | NA | | 4375 + 375 | | |
| 5 | 1 mg/mL | 375 | 2500 | 3375 | 1x DPBS, | 25 |
| | Ovalbumin + | | | | 1250 | |
| | W₈₀5EC | | | | | |
| 6 | 1 mg/mL | 375 | 2500 | 4375 | 1x DPBS, | 5 |
| | Ovalbumin + | | | | 250 | |
| | W₈₀5EC | | | | | |
| 7 | W₈₀5EC in water | 0 | 2500 | 5000 | NA | 0 |
| | without oval | | | | | |

Olvalbumin (Sigma A5503-5G Lot #118K7002), was mixed with water to generate a 2 mg/ml stock and stored at 4° C. 1× Dulbecco's Phosphate Buffered Saline (DPBS) without calcium and magnesium was obtained from MediaTech (Manassas, Va.) (148.82 mM). 60% W₈₀5EC, pH 5.40, was obtained from NanoBio Corporation, Ann Arbor, Mich.

Ovalbumin/W₈₀5EC emulsion mixtures were made with the buffers indicated in Table 11, starting with the addition, sequentially, of water, buffer, ovalbumin and emulsion into a 24 mL glass vial (total volume 7.5 mL). The ingredients were mixed by pipetting the liquid up and down several times.

The mixtures were then subjected to centrifugation in a fixed angle ultracentrifuge and spun at 30,000 g (20,000 rpm) for 60 minutes at room temperature (25 C).

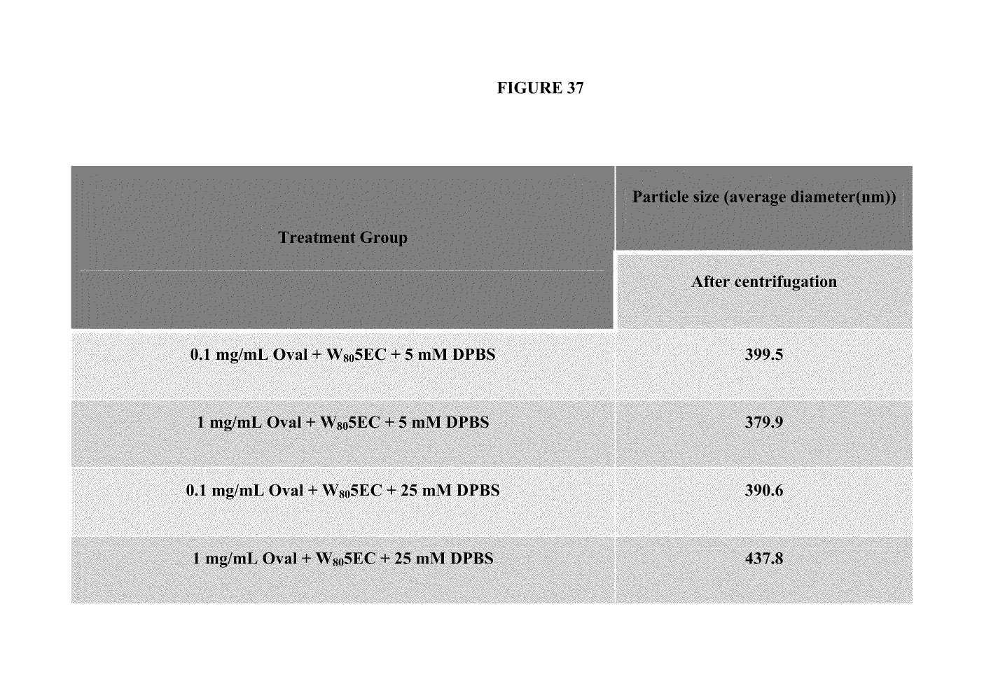

The average particle size (diameter) present in the ovalbumin/W₈₀5EC mixture post centrifugation is shown in FIG. 37.

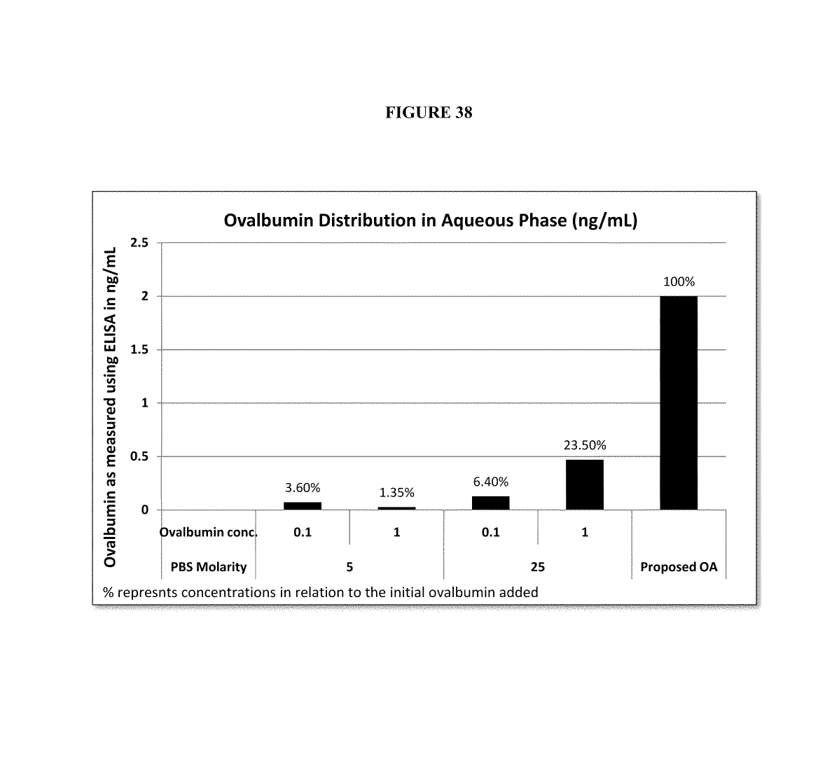

The aqueous phase of the centrifuged product was removed and analyzed using Chicken Egg Ovalbumin Kit 6050 (Alpha Diagnostic International, San Antonio, Tex.) following the manufacturer's instructions. The ovalbumin concentration present in the aqueous phase was calculated using the standards provided by the kit and the stock albumin used in the study. The aqueous phase ovalbumin concentration is shown in FIG. 38.

Characterization of ovalbumin present in the aqueous phase of the NE indicates that when using 5 mM PBS in a mixture, more than 96% ovalbumin was present in the oil phase (3.6% present in the aqueous phase) of the NE. For samples that had higher PBS molarity (25 mM), the ovalbumin in the oil phase was greater than 93% (6.4% present in the aqueous phase) when the starting concentration of the ovalbumin is 0.1 mg/mL and >76% when the ovalbumin concentration is 1 mg/mL

### Example 10

### Mucosal and Systemic Immune Responses Induced by Immunogenic Compositions of the Invention

### Immunogenicity Studies in Ferrets

A total of four ferret studies, investigating the W₈₀5EC-adjuvant, involving 354 animals were conducted. In these studies intranasal administration was accomplished in anesthetized ferrets using either a pipette or a tuberculin syringe and 18 gauge gavage needle. Doses were administered as drops, slowly into each nare.

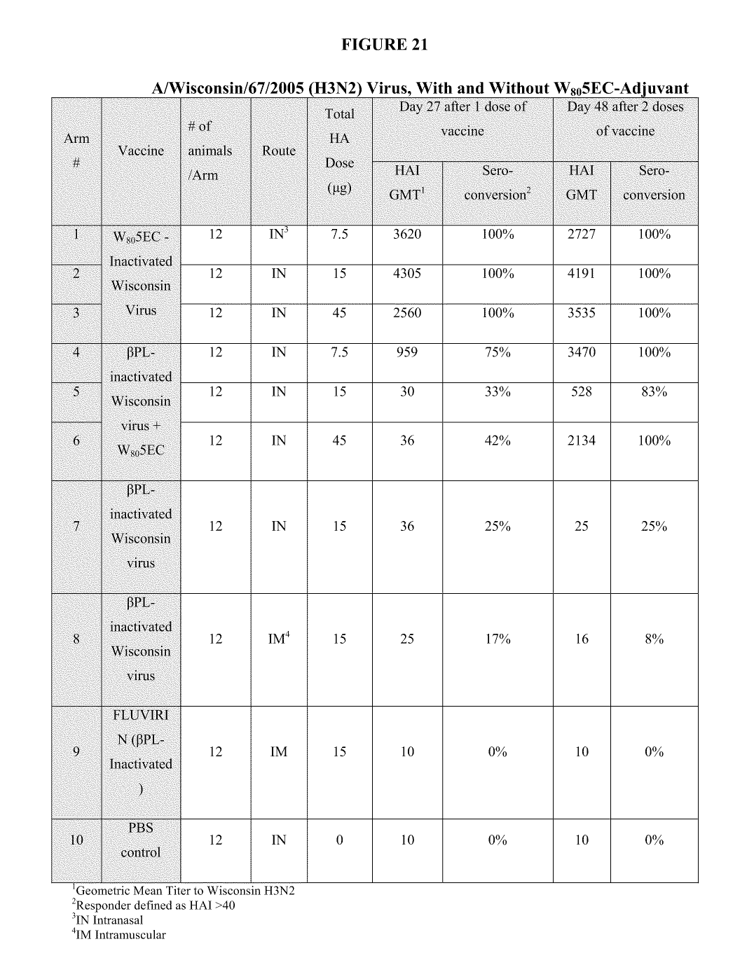

Ferret study #1 was an exploratory investigation to evaluate the adjuvant properties of the W₈₀5EC-adjuvant with whole influenza virus inactivated by various methods. The study design, vaccine doses, hemagglutination inhibition (HAI) geometric mean titers (GMT) and seroconversion rates are presented in FIG. 21.

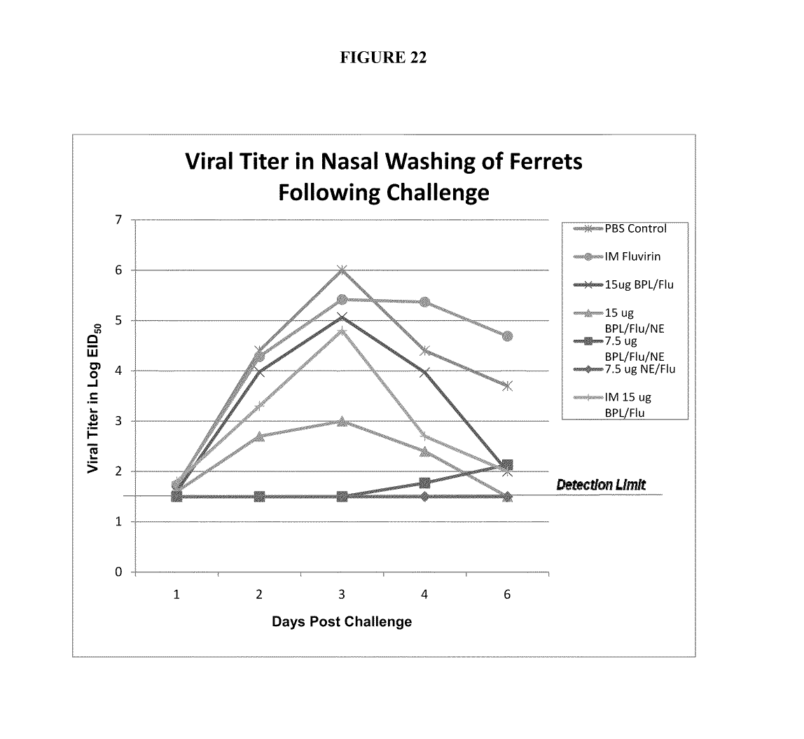

In this study, previously seronegative ferrets were challenged with 10⁶ EID₅₀ (Egg Infectious Dose₅₀) of A/Wisconsin/67/2005 (H3N2) virus on day 49 following vaccination with the same virus. Nasal washes were collected on days 1, 2, 3, 4 and 6 after challenge and titrated for viral concentration. The nasal washes from W₈₀5EC immunized ferrets had evidence of little or no virus compared with control ferrets that had significant amounts of virus (See FIG. 22).

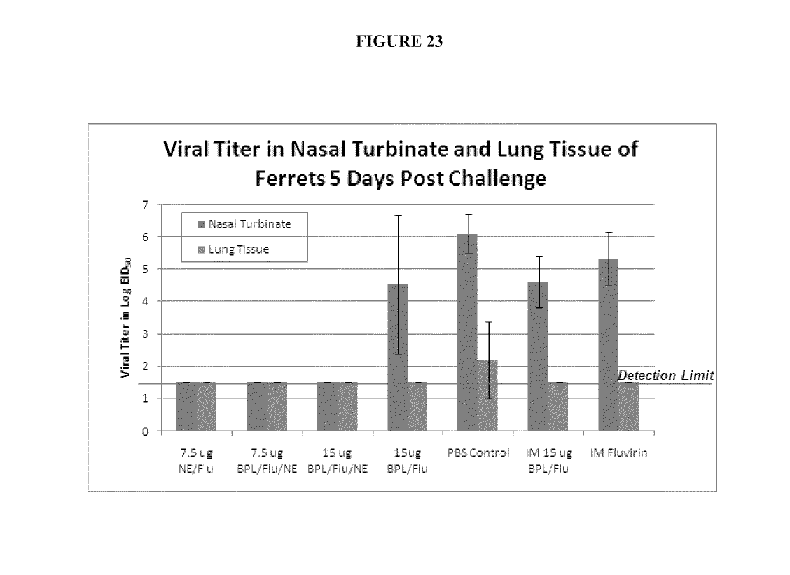

Subgroups of the ferrets were sacrificed on day 5 post challenge to determine viral load in the nasal turbinates and lung. While this influenza strain did not result in significant viral concentrations in the lungs of control animals, significant concentrations of virus were found in the nasal turbinates of control animals. The immunized animals, however, did not show evidence of virus in their nasal turbinates, indicating that they had developed sterilizing immunity (See FIG. 23).

In this study, the animals were observed twice daily for morbidity and mortality, and clinical signs, body weights and body temperatures were evaluated weekly. No significant clinical signs or effects on body weights or body temperature were noted and no animal was observed to have clinically abnormal signs or altered activity in this study, except as described below. One male ferret (#8507) immunized IM with 15 g of -propiolactone (PL) inactivated A/Wisconsin virus was euthanized after exhibiting weight loss over a two week period. Depression and dehydration were noted prior to euthanasia and the cause of death was considered unrelated to treatment with the test article.

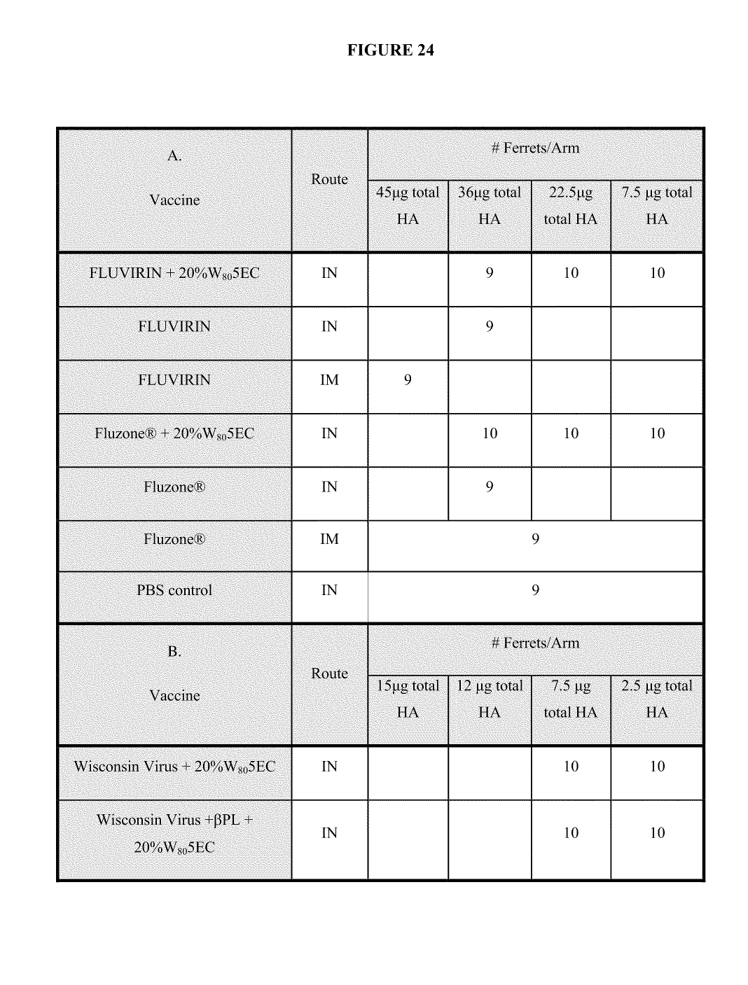

Ferret Study #2. Ferret study #2 was designed to examine immune responses to increasing total HA antigen doses of 7.5, 22.5 and 36 g mixed with 20% W₈₀5EC after one and two doses. HAI titers to A/Wisconsin (H3N2), A/Solomon Islands (H1N1) and B/Malaysia contained in the commercial Fluvirin® and FLUZONE (2007-2008) vaccines were evaluated (See FIG. 24A). Other arms of this study investigated vaccines prepared using whole inactivated A/Wisconsin virus mixed with 20% W₈₀5EC (See FIG. 24B).

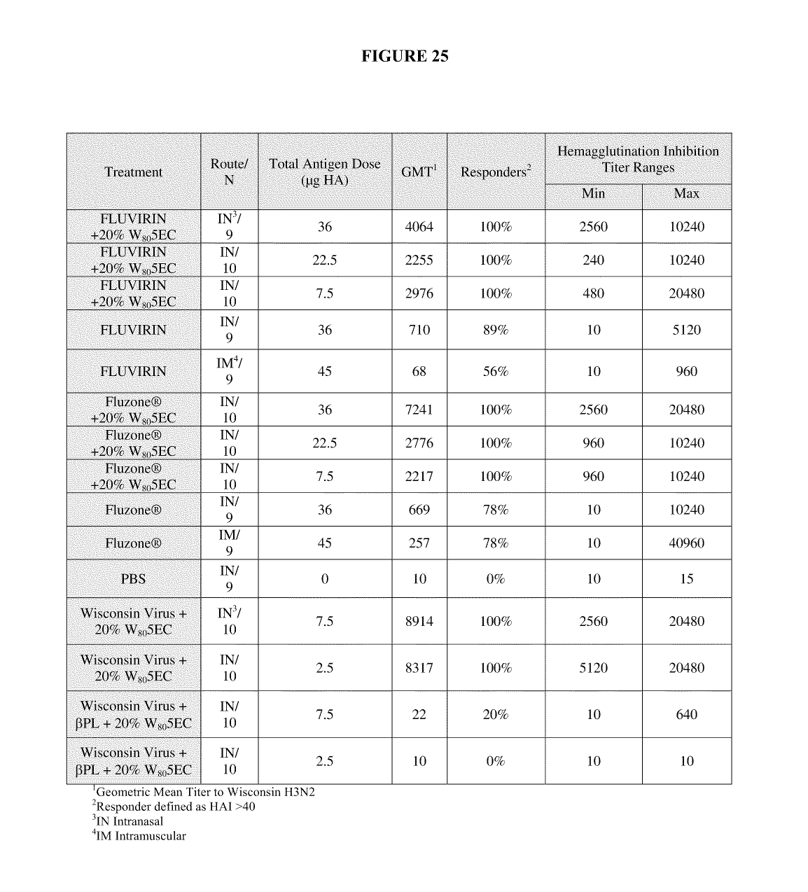

Doses of W₈₀5EC-adjuvanted commercial vaccines as low as 7.5 g total HA antigen resulted in geometric mean titers (GMT)>2200 for A/Wisconsin (H3N2) following a single vaccination in naïve male ferrets (See FIG. 25). This represents a >220-times increase from baseline and a 32-times increase compared to Fluvirin® IM and >8-times increase compared to FLUZONE IM. Notably, all ferrets given Fluvirin® or FLUZONE+20% W₈₀5EC responded with HAI titers >40 (See FIG. 25).

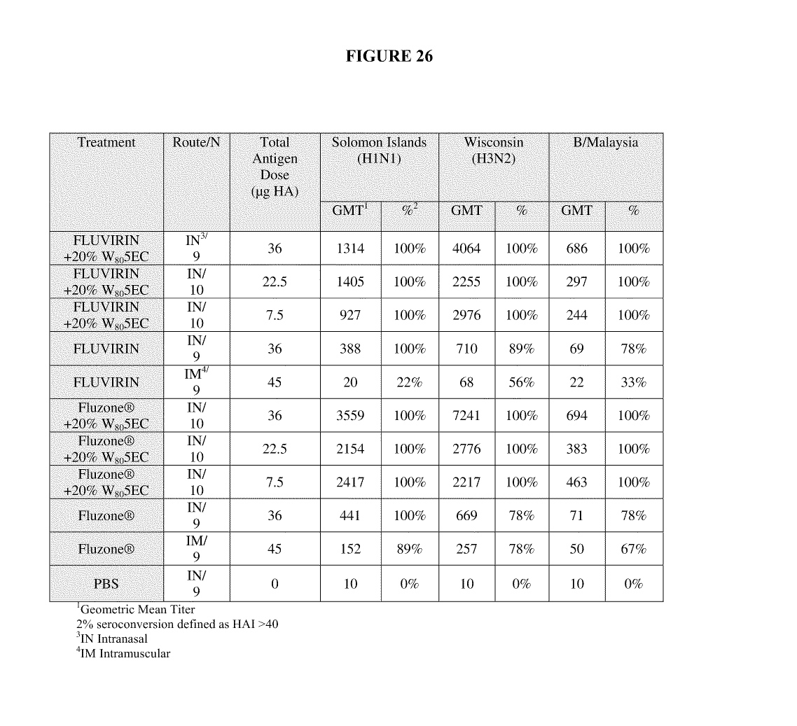

In study #2, HAI titers to antigens for A/Solomon Islands (H₁N₁) and B/Malaysia contained in the W₈₀5EC-adjuvanted commercial vaccines were also determined and demonstrated ≈100-times increase and ≧U 25-times increase relative to baseline, respectively. In addition, for Solomon Islands there was a >40-times increase in the adjuvanted arm compared to Fluvirin® IM and >13-times increase compared to FLUZONE IM. For B/Malaysia there was a >11-times increase compared to Fluvirin® IM and a >7 times increase compared to FLUZONE IM (See FIG. 26).

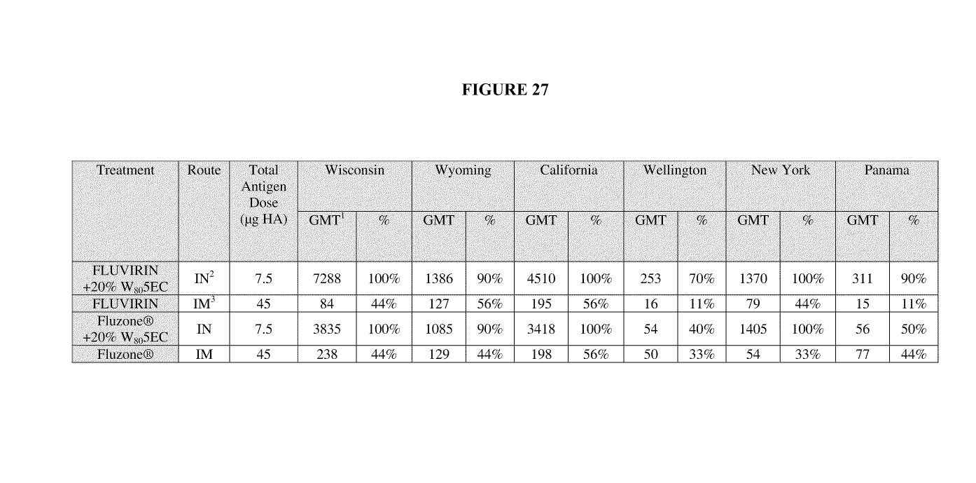

In ferret study #2, HAI titers to H₃N₂ strains not contained in the commercial vaccines were determined on Day 48 following two intranasal vaccine doses (Day 0 and Day 28) and are summarized in FIG. 27. The 20% W₈₀5EC-adjuyanted vaccines elicited 25-times to 720-times increases from baseline HAI titers and significant (≧70%) seroconversion to all strains tested for Fluvirin®+20% W₈₀5EC and all strains except Wellington and Panama in animals receiving FLUZONE+20% W₈₀5EC. The IM control groups had significantly lower rates of seroconversion.

In addition, in study #2, ferrets that received 7.5 total μg HA antigen (FLUZONE or Fluvirin®) with 20% W₈₀5EC were challenged with 10H^{U7UH}EID₅₀ of A/Wisconsin (H3N2) strain. These ferrets did not show evidence of virus in their nasal washes on days 2-6 following the challenge.

In this study, the animals were observed twice daily for morbidity and mortality and no abnormal observations or altered activities were noted. In addition, clinical signs, body weights and body temperatures were evaluated weekly. No significant clinical signs or changes in body weights or temperatures were reported.

Ferret Study #3. Ferret study #3 was designed to further explore antigen-sparing activity and cross-reactivity following a single intranasal 20% W₈₀5EC-adjuyanted vaccine dose. The lowest total antigen dose from study #2 (7.5 g total antigen) was replicated and antigen-sparing activity was assessed by administration of lower doses of 3 and 0.9 μg total antigen. All 20% W₈₀5EC-adjuvanted commercial vaccine doses elicited immune responses that were significantly enhanced when compared to an intramuscular control that received 37.5 μg FLUZONE (See FIG. 28).

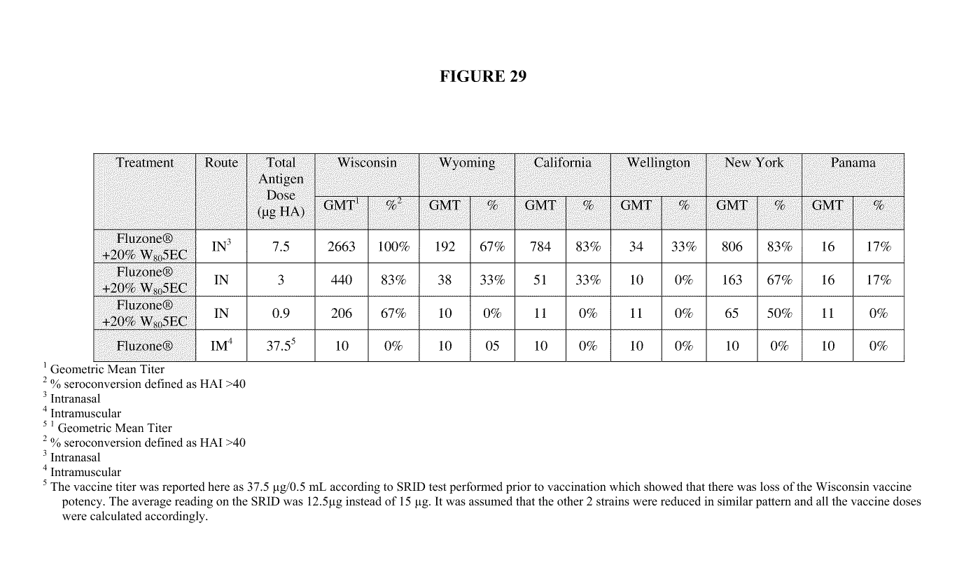

In ferret study #3, HAI titers for A/Wisconsin and other H₃N₂ influenza strains not contained in the commercial vaccine were determined and robust immune responses at a total antigen dose of 7.5 μg were demonstrated (See FIG. 29), with less robust cross-reactivity at lower antigen concentrations and for Wellington and Panama following a single vaccination at the 7.5 g dose.

In ferret study #3, the animals were observed twice daily for morbidity and mortality and no abnormal observations or altered activity was noted. In addition, clinical signs, body weights and body temperatures were evaluated weekly. No significant clinical signs or effects on body weights or body temperatures were reported in this study. There were no deaths in this study.

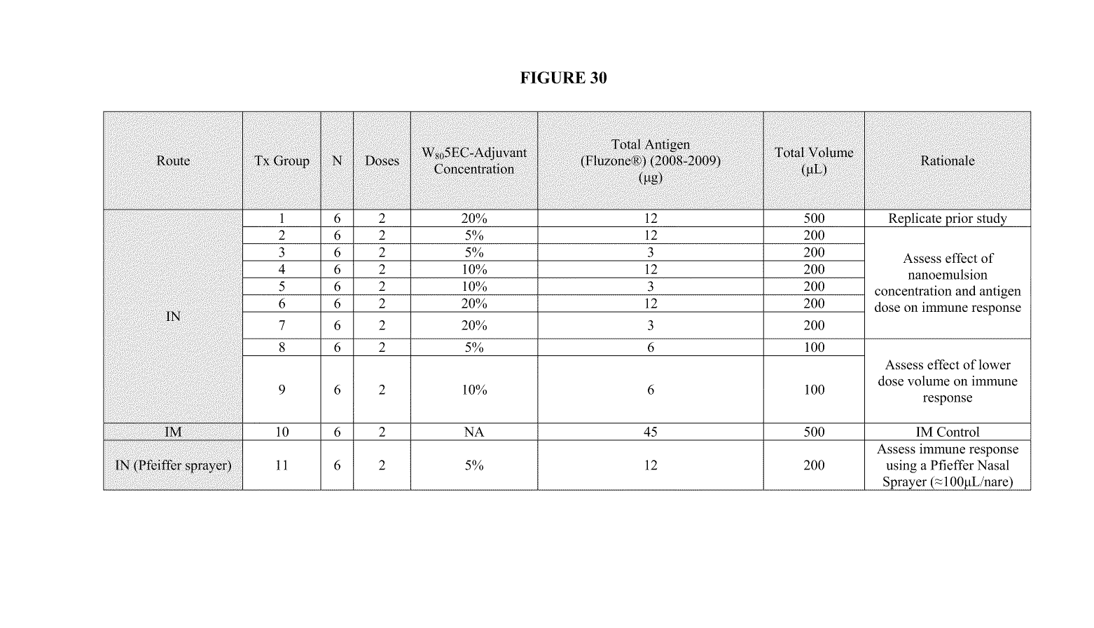

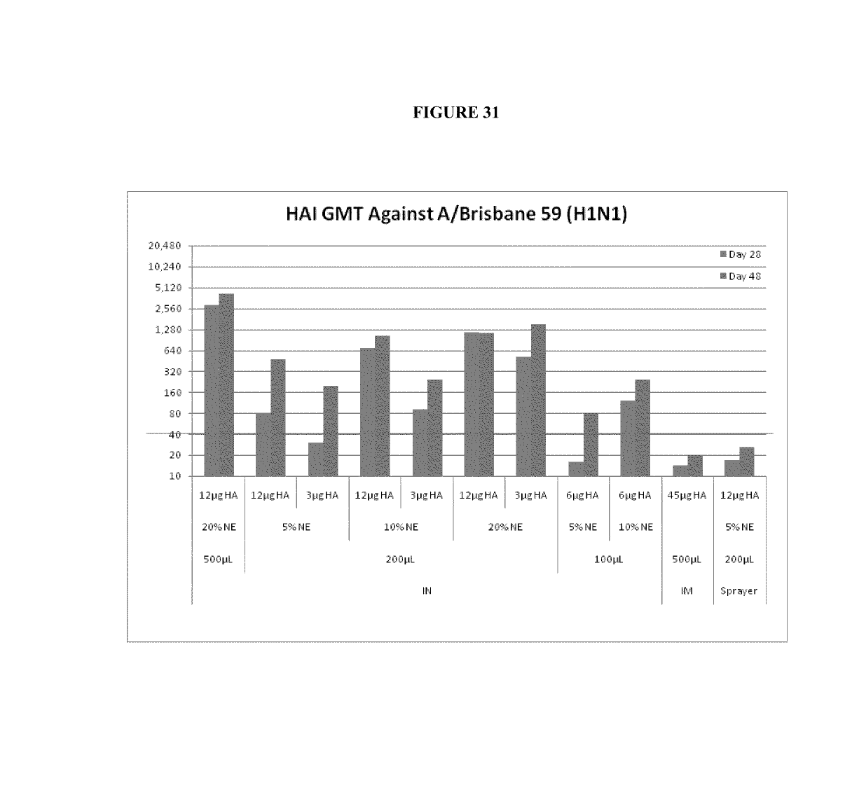

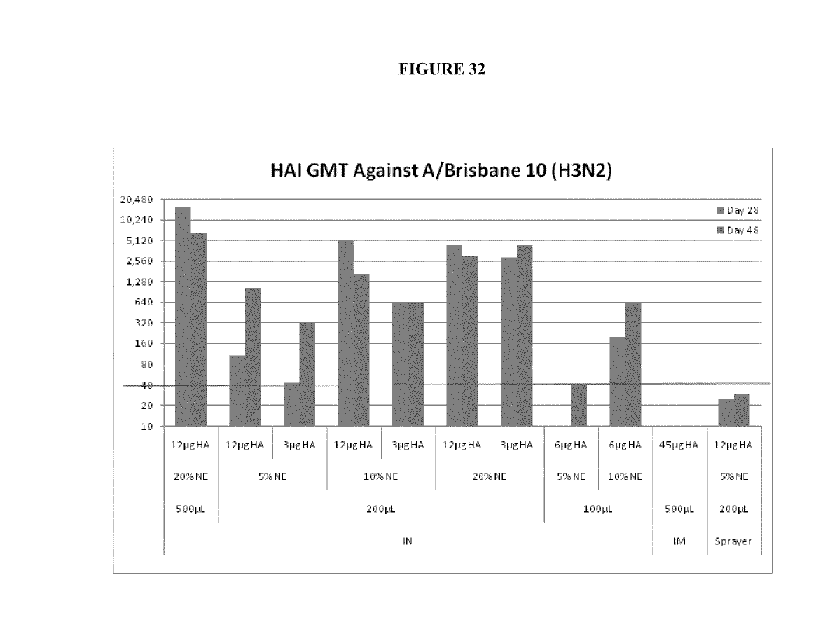

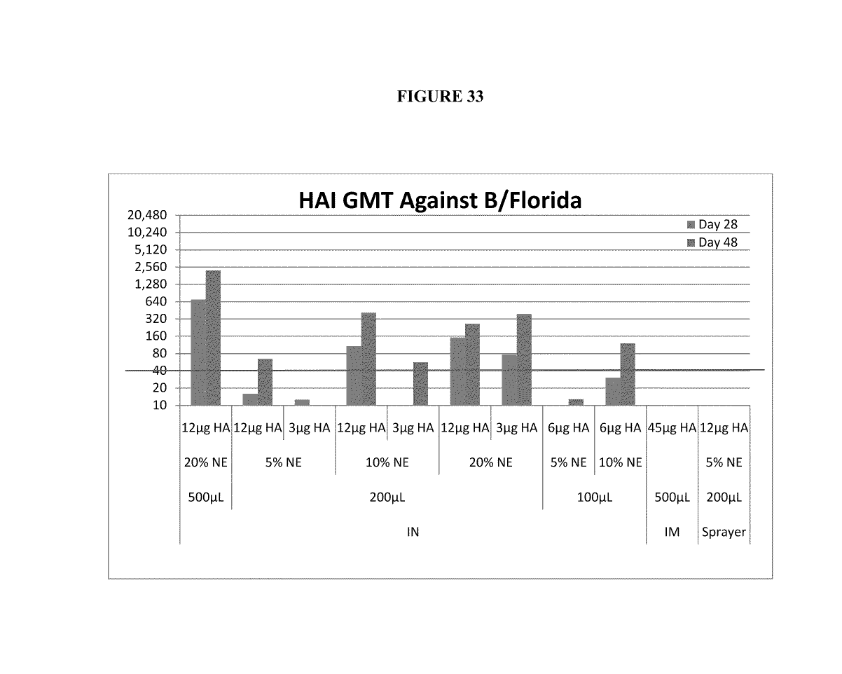

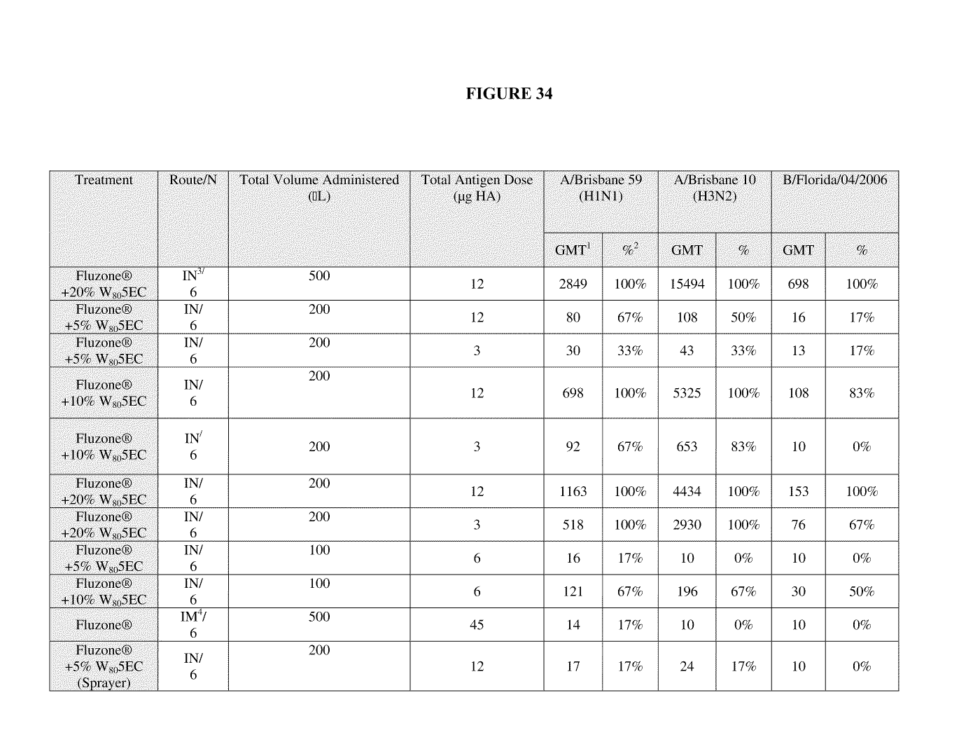

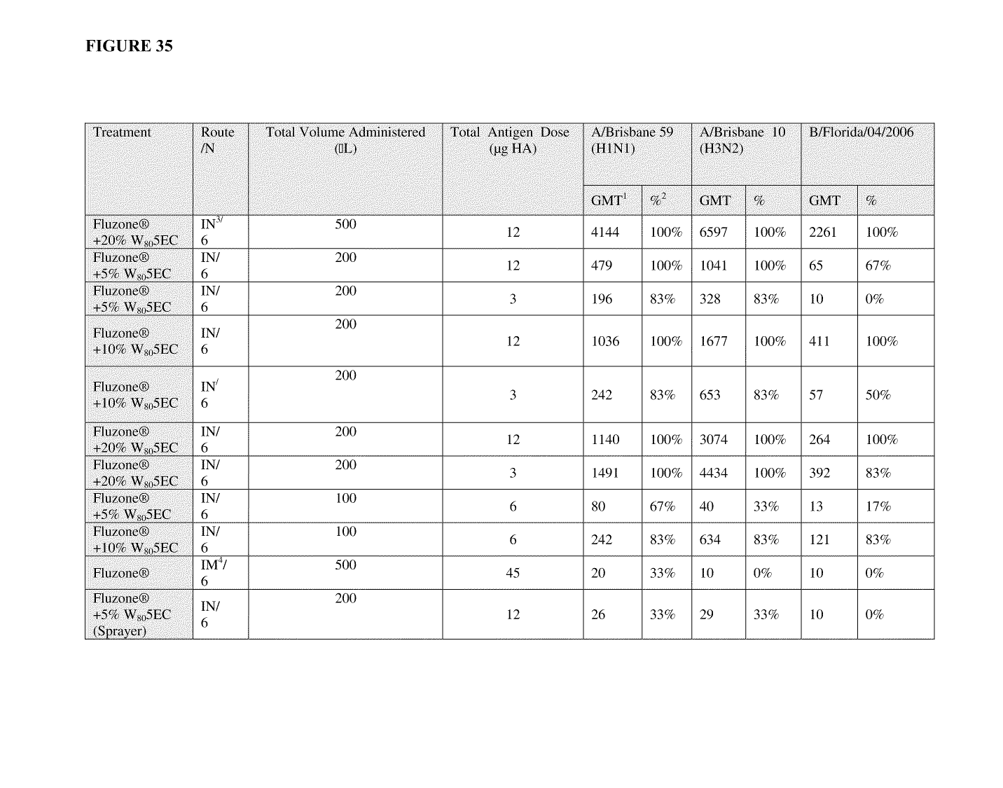

Ferret Study #4. This study was designed to determine the effect of different W₈₀5EC-adjuvant concentrations on the immune response at equivalent antigen doses, to examine the effect of dose volume and to assess the potential utility of using a Pfeiffer nasal sprayer device. The design is summarized in FIG. 30 and data are displayed graphically (See, e.g., FIGS. 31-33) and in tables shown in FIGS. 34 and 35).

As shown in the figures, the immune response to FLUZONE 12 g+20% W₈₀5EC-adjuvant administered at 500 L was very similar to the prior experience obtained in earlier studies. Increasing the W₈₀5EC-adjuvant concentration from 5% to 10% or to 20% at an equivalent antigen dose resulted in increased GMT response and increased seroconversion at 3, 6 and 12 g total HA doses, respectively. Thus, the present invention provides that increasing the volume from 200 L to 500 L at an equivalent 12 g total HA dose also increased the immune response. The IM control elicited a minimal immune response and performed as expected based upon prior experience. Administration of vaccine using the Pfeiffer sprayer did not elicit a significant immune response and could indicate the difficulty in delivering the vaccine to the anatomic area important in generating an optimal immune response.

Robust immune responses were demonstrated after one and two vaccinations using 10% W₈₀5EC- and 20% W₈₀5EC-adjuvant each with 12 μg total HA.

In ferret study #4, the animals were observed twice daily for morbidity and mortality and no abnormal observations or altered activity was noted. In addition, clinical signs, body weights and body temperatures were evaluated weekly. No significant clinical signs or effects on body weights or body temperatures were reported in this study. There were no deaths in this study. Thus, the invention provides that intranasal administration of W₈₀5EC-adjuvanted vaccines was well tolerated without significant treatment-related clinical abnormalities.

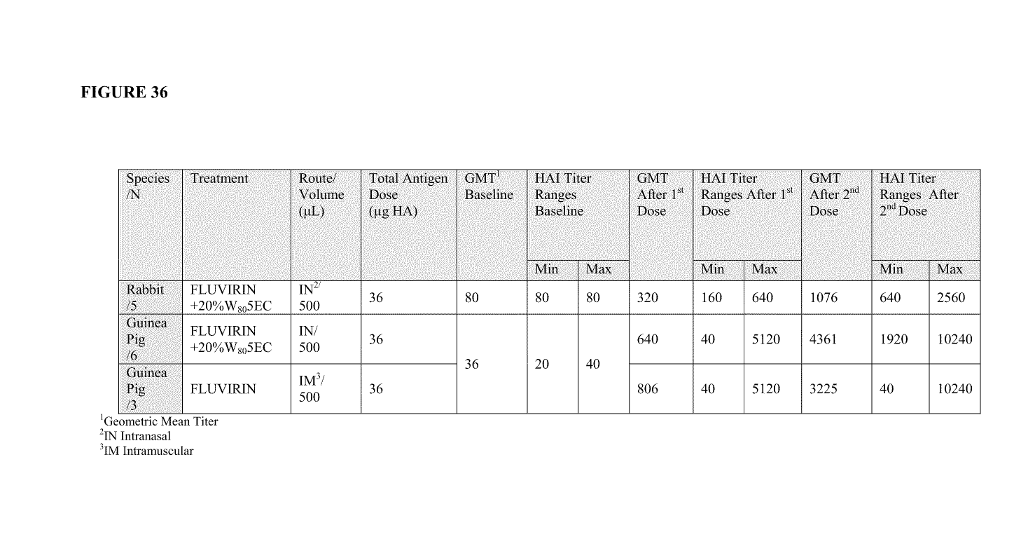

Studies in Rabbits and Guinea Pigs. Experiments were conducted during development of embodiments of the invention to characterize immune responses in rabbits and guinea pigs (See Figure non-GLP immunogenicity study (FIG. 36).

Rabbits and guinea pigs were administered two doses of 20% W₈₀5EC-adjuvant with Fluvirin® (36 μg total HA antigen dose) intranasally to determine immune responsiveness. Rabbits and guinea pigs developed A/Wisconsin strain specific antibodies >13-times and >120-times baseline values, respectively (See FIG. 36). Guinea pigs administered Fluvirin® IM (36 g total antigen dose) developed A/Wisconsin strain specific antibodies >90-times baseline values.

One of the 5 rabbits (#4) had reduced food consumption and weight loss and was euthanized 37 days after receiving the second intranasal vaccination of Fluvirin® 36 g+20% W₈₀5EC-adjuvant. Gross pathologic examination was unremarkable and the animal appeared thin but healthy on gross examination. Therefore, no histopathologic examinations were performed.

In this exploratory study, two of the 6 guinea pigs died after intranasal administration of Fluvirin® 36 g+20% W₈₀5EC-adjuvant. The first animal was examined and found to be healthy at 24 and 48 hours following the first vaccination, but was found dead day three after vaccination. Histopathologic examination was not conducted due to extensive autolysis; thus, the cause of death could not be assessed. The second guinea pig died one day after the second intranasal vaccination and had grossly abnormal lungs on necropsy. Microscopic examination revealed severe, acute multifocal pulmonary edema. The cause of death was attributed to respiratory arrest secondary to acute pulmonary edema. A sentinel guinea pig housed in the same room, was found to be adenovirus positive.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

What is claimed is:

1. An immunogenic composition comprising a nanoemulsion, wherein the nanoemulsion comprises (i) an aqueous phase; (ii) an oil phase; and (iii) one or more immunogens; wherein the one or more immunogens reside within the oil phase of the emulsion.

2. The immunogenic composition of claim 1, wherein the nanoemulsion further comprises (iv) one or more additional compounds.

3. The immunogenic composition of claim 2, wherein one or more additional compounds are selected from the group consisting of organic solvent, a non-ionic, anionic or cationic surfactant, quaternary ammonium containing compound, cationic halogen containing compound, germination enhancer, interaction enhancer, and pharmaceutically acceptable carrier compound.

4. The immunogenic composition of claim 3, wherein the nanoemulsion comprises oil, cationic surfactant, water and an organic solvent.

5. The immunogenic composition of claim 1, wherein the composition is more immunogenic than an equal amount of immunogen mixed with an emulsion wherein the immunogen does not reside within the oil phase of the emulsion.

6. The immunogenic composition of claim 1, wherein the composition does not contain a detectable level of a compound selected from the group consisting of a bacterial toxin, an endotoxin, and a cytokine.

7. The immunogenic composition of claim 1, wherein the oil phase comprises an oil selected from the group consisting of soybean oil, avocado oil, olive oil, canola oil, corn oil, rapeseed oil, safflower oil, sunflower oil, fish oil, squalene, and water insoluble vitamins.

8. The immunogenic composition of claim 1, wherein the organic solvent facilitates solvation of the immunogen into the oil phase of the emulsion.

9. The immunogenic composition of claim 3, wherein the organic solvent is selected from the group consisting of ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and a combination thereof.

10. The immunogenic composition of claim 3, wherein the surfactant is a polysorbate (TWEEN) selected from the group consisting of polysorbate 20 (TWEEN 20), polysorbate 60 (TWEEN 60) and polysorbate 80 (TWEEN 80).

11. The immunogenic composition of claim 3, wherein the cationic halogen containing compound is selected from the group consisting of cetylpyridinium halides, cetylpyridinium chloride, cetyltrimethylammonium halides, cetyldimethylethylammonium halides, cetyldimethylbenzylammonium halides, cetyltributylphosphonium halides, dodecyltrimethylammonium halides, and tetradecyltrimethylammonium halides.

12. The immunogenic composition of claim 1, wherein the nanoemulsion comprises 5 vol. % of polysorbate 80 (TWEEN 80), about 8 vol. % of ethanol, about 1 vol. % of cetylpyridinium chloride (CPC), about 64 vol. % of soybean oil, and about 22 vol. % of deionized water.

13. The immunogenic composition of claim 1, wherein the one or more immunogens is a pathogen or pathogen product selected from the group consisting of protein, peptide, polypeptide, nucleic acid, polysaccharide, membrane component derived from the pathogen, and inactivated pathogen.

14. The immunogenic composition of claim 1, wherein the one or more immunogens is an influenza immunogen.

15. The immunogenic composition of claim 1, wherein the one or more immunogens comprise a commercially available influenza vaccine.

16. A method of inducing an immune response to an immunogen, comprising:
a) providing: an immunogenic composition comprising (i) a nanoemulsion, wherein the nanoemulsion comprises: 1. oil; 2. ethanol; 3. a surfactant; 4. a quaternary ammonium compound; and 5. distilled water; and (ii) one or more immunogens; wherein the one or more immunogens reside within the oil phase of the emulsion; and
b) administering the immunogenic composition to a subject under conditions such that the subject produces an immune response to the immunogen, wherein the administering comprises contacting the immunogenic composition with a mucosal surface of the subject.

17. The method of claim 16, wherein the one or more immunogens is a pathogen or pathogen product selected from the group consisting of protein, peptide, polypeptide, nucleic acid, polysaccharide, membrane component derived from the pathogen, and inactivated pathogen.

18. The method of claim 16, wherein the one or more immunogens is selected from the group consisting of virus, bacteria, fungus and pathogen products derived from the virus, bacteria, or fungus.

19. The method of claim 18, wherein the virus is selected from the group consisting of influenza A virus, influenza B virus, avian influenza virus, H5N1 influenza virus, H1N1 influenza virus, West Nile virus, SARS virus, Marburg virus, Arenaviruses, Nipah virus, alphaviruses, filoviruses, herpes simplex virus I, herpes simplex virus II, paramyxovirus, respiratory synthetial virus, sendai virus, sindbis virus, vaccinia virus, parvovirus, human immunodeficiency virus, hepatitis B virus, hepatitis C virus, hepatitis A virus, cytomegalovirus, human papilloma virus, picornavirus, hantavirus, junin virus, and ebola virus.

20. The method of claim 18, wherein the bacteria is selected from the group consisting of Bacillus cereus, Bacillus circulars and Bacillus megaterium, Bacillus anthracis, Brucella, Vibrio cholera, Coxiella burnetii, Francisella tularensis, Chlamydia psittaci, Ricinus communis, Rickettsia prowazekii, bacteria of the genus Salmonella, Cryptosporidium parvum, Burkholderia pseudomallei, Clostridium perfringens, Clostridium botulinum, Vibrio cholerae, Streptococcus pyogenes, Streptococcus agalactiae, Streptococcus pneumonia, Staphylococcus aureus, Neisseria gonorrhea, Haemophilus influenzae, Escherichia coli, Salmonella typhimurium, Shigella dysenteriae, Proteus mirabilis, Pseudomonas aeruginosa, Yersinia pestis, Yersinia enterocolitica, and Yersinia pseudotuberculosis.

21. The method of claim 18, wherein the one or more immunogens comprise immunogenic protein present in a commercially available vaccine.

22. The method of claim 16, wherein the amount of immunogen present in the immunogenic composition is selected from the group consisting of an amount less than one half the amount of immunogen present in a commercially available vaccine, an amount equal to the amount of immunogen present in a commercially available vaccine, an amount that is 90% of the amount of immunogen present in a commercially available vaccine, an amount that is 75% of the amount of immunogen present in a commercially available vaccine, an amount that is 60% the amount of immunogen present in a commercially available vaccine, an amount that is 33% the amount of immunogen present in a commercially available vaccine, and an amount that is 25% the amount of immunogen present in a commercially available vaccine.

23. The method of any one of claims 20-22, wherein the immune response elicited by the immunogenic composition is equal to or greater than the immune response elicited by the full dose amount of immunogen present in the commercially available, seasonal influenza vaccine administered alone.

24. The method of claim 16, wherein the immunogen comprises a pathogen product.

25. The method of claim 16, wherein the administering produces a mucosal immune response against the immunogen.

26. The method of claim 16, wherein the administering comprises intranasal administration.

27. The method of claim 16, further comprising repeating step b) administering the immunogenic composition to the subject.

28. The method of claim 16, wherein the subject exhibits a higher titer of immunogen-specific antibodies relative to a subject not administered the immunogenic composition.

29. The method of claim 16, wherein the subject exhibits a higher titer of immunogen-specific antibodies relative to a subject administered the one or more immunogens not residing within the oil phase of the emulsion.

30. The method of claim 28 or claim 29, wherein said immunogen-specific antibodies comprise IgG antibodies.

31. The method of claim 28 or claim 29, wherein said immunogen-specific antibodies comprise IgA antibodies.

32. An emulsion comprising oil droplets with a mean diameter of less than 1 micron, said emulsion comprising: an oil, a cationic detergent, a solvent, a nonionic detergent, and an antigen, wherein,
(i) said antigen is contained within the oil phase of the emulsion for delivery into antigen-presenting cells,
(ii) said solvent facilitates the localization of the antigen within the oil phase of the emulsion, and,
(iii) said nonionic detergent stimulates the activity of immune-presenting cells, for purposes of inducing an immune response in a host animal.
